(19) United States
(12) Patent Application Publication
(10) Pub. No.: US 2007/0032453 A1
(43) Pub. Date: Feb. 8, 2007

(54) ADJUVANT CHEMOTHERAPY FOR ANAPLASTIC GLIOMAS

(75) Inventors: Rheal A. Towner, Piedmont, OK (US); Yashige Kotake, Oklahoma City, OK (US); Robert A.. Floyd, Oklahoma City, OK (US)

Correspondence Address: FULBRIGHT & JAWORSKI L.L.P., 600 CONGRESS AVE., SUITE 2400, AUSTIN, TX, 78701, US

(73) Assignee: Oklahoma Medical Research Foundation

(21) Appl. No.: 11/460,866

(22) Filed: Jul. 28, 2006

Related U.S. Application Data
(60) Provisional application No. 60/705,721, filed on Aug. 4, 2005.

(51) Int. Cl. A61K 31/7008; A61K 31/13

(52) U.S. Cl. 514/62; 514/645

(57) ABSTRACT

The present invention involves the use of nitrone free radical trapping agents in the treatment and prevention of gliomas. The agents may be used alone or combined with other traditional chemo- and radiotherapies and surgery, to treat or prevent glioma occurrence, recurrence, spread, growth, metastasis, or vascularization.

This application claims benefit of priority to U.S. Provisional Application Ser. No. 60/705,721, filed Aug. 4, 2005, the entire contents of which are hereby incorporated by reference.

The government owns rights in the present invention pursuant to Grant No. 2 P20 RR016478 from the National Institutes of Health.

### BACKGROUND OF THE INVENTION

1. Field of the Invention

The present invention relates generally to the fields of oncology and chemotherapy. More particularly, it concerns the combined use of antioxidants to treat gliomas.

2. Description of Related Art

Gliomas are a diverse group of brain tumors that arise from normal “glial” cells of the brain and/or their precursor cells. The most important determinant of survival for gliomas is the “grade” of the glioma. Secondary determinants of survival are age at diagnosis, performance status, and extent of surgery. Patients with low-grade gliomas have a protracted natural history with generally long survival times, while those with high grade gliomas are much more difficult to successfully treat and have shorter survival times. All gliomas have specific signs and symptoms that are primarily related to the location and size of the glioma.

The temporal lobe gliomas, for example, may cause seizures, difficulty with speech and/or loss of memory. The frontal lobe gliomas may cause seizures, behavioral changes, weakness of the arms or legs on the opposite side of the body, and/or difficulty with speech. The occipital gliomas may cause loss of vision. The parietal gliomas may cause loss of spatial orientation, diminished sensation on the opposite side of the body, and/or inability to recognize once familiar objects or persons.

Astrocytomas are glioma tumors that arise from brain cells called astrocytes or their precursors. Astrocytes are cells in the central nervous system that support neuronal function. Astrocytomas can be graded by histologic features that signify increasing malignancy into astrocytoma, anaplastic astrocytoma, or glioblastoma multiforme. Anaplastic astrocytoma and glioblastoma multiforme are considered high-grade gliomas while the astrocytoma is considered to be a low-grade glioma. High-grade tumors grow rapidly and can easily infiltrate and spread through the brain. Low-grade astrocytomas can also infiltrate the brain but are usually more localized and grow slowly over a long period of time. High-grade tumors are much more aggressive and require very intense therapy. The majority of astrocytic tumors in children are low-grade, whereas the majority in adults are high-grade. Astrocytomas can occur anywhere in the brain and spinal cord, however the majority are located in the cerebral hemispheres.

Oligodendrogliomas are also gliomas. They arise from oligodendrocytes and/or their cell precursors. Normal oligodendrocytes provide myelin, a fatty substance that covers nerve axons in the brain and spinal cord and allows nerves to conduct electrical impulses more efficiently. Oligodendrogliomas are classified as low grade oligodendroglioma (less aggressive) and anaplastic oligodendroglioma (more aggressive). More common than pure oligodendrogliomas are low grade and anaplastic tumors that are a mixture of astrocytoma and oligodendroglioma (“oligoastrocytomas”).

Anaplastic oligodendrogliomas and mixed oligoastrocytomas are more sensitive to cytotoxic chemotherapy than astrocytomas. A high rate of response to the PCV (procarbazine (matulane), CCNU (lomustine), vincristine) chemotherapy has made the use of this regimen, if not the standard of care for these tumors, at least a very common treatment. Low grade oligodendrogliomas are also sensitive to chemotherapy, and PCV can be used when low grade tumors begin to grow despite prior surgery/radiation therapy.

In 1983, it was reported that surgery plus radiation therapy and BCNU chemotherapy significantly improved the survival of patients with malignant glioma, as compared to those treated with surgery plus radiation therapy without chemotherapy. In one study, both procarbazine and streptozotocin demonstrated effectiveness similar to that of BCNU. Other studies showed that BCNU alone is as effective as BCNU followed by procarbazine, and that PCNU was no better than BCNU. In some studies the PCV combination was found superior to BCNU for anaplastic gliomas, while in other studies they are considered equivalent.

Another approach involves the combined use of DFMO-PCV in treating anaplastic gliomas, extending the study of Levin et al. (2000). Surprisingly, and in marked contrast to the results seen with glioblastoma multiforme, the combination of DFMO and PCV significantly increased the overall survival of patients with anaplastic gliomas. It is possible that a feature distinguishing tumor response to DFMO is the level and activity of the enzyme omithine decarboxylase (ODC). It is likely that one factor that distinguishes DFMO benefit is the tumor level of ODC; patients with relatively lower levels of ODC appear to respond better to DFMO and DFMO-nitrosourea combinations. This conclusion is based on published observations that show that (1) ODC levels are directly correlated with malignant grade of glioma (Scalabrino et al., 1982; Scalabrino and Ferioli, 1985; Emestus et al, 1992; Emestus et al., 1996); (2) the fact that DFMO (+/− methylglyoxal bisguanylhydrazone) activity was not seen in glioblastoma multiforme and best seen in mid-grade anaplastic gliomas (Levin et al., 1995; Levin et al., 1992) with lower ODC levels; and (3) in combination with a nitrosourea, activity was not seen in glioblastoma multiforme and best seen in mid-grade anaplastic gliomas (Prados et al., 1989; Levin et al., 2000) with lower ODC levels.

In addition to controlled survival-based clinical trials, a large number of agents have also been tested in response-based studies in glioma patients. To date, however, no drug has been found to be more effective than the nitrosoureas. Thus, there is a need for effective and improved therapies for anaplastic gliomas.

### SUMMARY OF THE INVENTION

Thus, in accordance with the present invention, there is provided a method for inhibiting the vascularization, growth or spread of a glioma comprising administering to a human subject with glioma a dose of a nitrone free radical trapping agent effective to inhibit the growth of said glioma. The nitrone free radical trapping agent may be an N-alkyl nitrone free radical trapping agent, such as phenyl butylnitrones, including phenyl N-tert-butylnitrone, 3-hydroxyphenyl N-tert-butylnitrone, 2-hydroxyphenyl N-tert-butylnitrone, 2-sulfoxyphenyl N-tert-butylnitrone or 4-hydroxyphenyl N-tert-butylnitrone. Also encompassed are modified butyl nitrone compounds such as 5,5-dimethyl-1-pyrroline-N-oxide and alpha(4-pyridyl-1-oxide)-N-tert-butyl nitrone. The method human subject may have a recurrent or metastatic glioma, or have previously failed one or more anti-glioma therapies. The effective dose may be from about 5 to about 150 mg/kg body weight per day. Administering may be through dietary administration, such as through supplementation of a food component. The effective amount is from about 0.005 w/w % to about 0.1 w/w % of the diet being administered. The glioma may be an astrocytoma, an oligodendroglioma, or a glioblastoma multiforme.

The method may further comprise measuring inducible nitric oxide synthase (iNOS) levels in cells of said glioma., such as using MRI using a labeled anti-iNOS antibody. The method may also further comprise measuring nitric oxide (NO) levels in tissues of said glioma, such as by measuring comprises MRI using a NO spin trapping agent, e.g., N-methyl-D-glucamine dithiocarbamate (MGD)-Fe(II)-NO complex. The method may also further comprise a secondary anti-glioma therapy, such as chemotherapy, including is lomustine, vincristine, matulane, PCV, BCNU, CCNU and/or DFMO, radiation or surgery.

In another embodiment, there is provided a method for inhibiting glioma development comprising (a) identifying a human subject at risk of developing a glioma and (b) administering to said subject a dose of a nitrone free radical trapping agent effective to inhibit the development of said glioma. The nitrone free radical trapping agent may be an N-alkyl nitrone free radical trapping agent, such as phenyl butylnitrones, including phenyl N-tert-butylnitrone (PBN), 3-hydroxyphenyl N-tert-butylnitrone, 2-hydroxyphenyl N-tert-butylnitrone, 2-sulfoxyphenyl N-tert-butylnitrone, 4-hydroxyphenyl N-tert-butylnitrone and PBN derivatives and analogs. Also encompassed are modified butyl nitrone compounds such as 5,5-dimethyl-1-pyrroline-N-oxide and alpha(4-pyridyl-1-oxide)-N-tert-butyl nitrone. The human subject may have a familial history of cancer or may have been exposed to a carcinogenic environment. Specific glioma risk factors include exposure to N-nitroso compounds or X-irradiation. The effective dose is from about 5 to about 150 mg/kg body weight per day. Administering may be through dietary administration, such as through supplementation of a food component, oral administration in the form of a pill or in liquid form, or via intravenous injection. The effective amount may be from about 0.005 w/w % to about 0.1 w/w % of the diet being administered. The glioma may be an astrocytoma, an oligodendroglioma, or a glioblastoma multiforme.

In yet another embodiment, there is provided a method for inhibiting glioma recurrence comprising administering to a subject previously having a glioma a dose of a nitrone free radical trapping agent effective to inhibit the development of said glioma. The nitrone free radical trapping agent may be an N-alkyl nitrone free radical trapping agent, such as phenyl butylnitrones, including phenyl N-tert-butylnitrone (PBN), 3-hydroxyphenyl N-tert-butylnitrone, 2-hydroxyphenyl N-tert-butylnitrone, 2-sulfoxyphenyl N-tert-butylnitrone, 4-hydroxyphenyl N-tert-butylnitrone and PBN derivatives and analogs. Also encompassed are modified butyl nitrone compounds such as 5,5-dimethyl-1-pyrroline-N-oxide and alpha(4-pyridyl-1-oxide)-N-tert-butyl nitrone. The glioma may be an astrocytoma, an oligodendroglioma, or a glioblastoma multiforme. The effective dose may be from about 5 to about 150 mg/kg body weight per day. Administering may be through dietary administration, such as through supplementation of a food component, oral administration in the form of a pill or in liquid form, or via intravenous injection. The effective amount is from about 0.005 w/w % to about 0.1 w/w % of the diet being administered.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

The terms “comprise” (and any form of comprise, such as “comprises” and “comprising”), “have” (and any form of have, such as “has” and “having”), “contain” (and any form of contain, such as “contains” and “containing”), and “include” (and any form of include, such as “includes” and “including”) are open-ended linking verbs. As a result, a device or a method that “comprises,” “has,” “contains,” or “includes” one or more elements possesses those one or more elements, but is not limited to possessing only those one or more elements or steps. Likewise, an element of a device or method that “comprises,” “has,” “contains,” or “includes” one or more features possesses those one or more features, but is not limited to possessing only those one or more features.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1I—T2-weighted images of C6 gliomas. Control rat at day 10 (FIG. 1A), day 18 (FIG. 1B) and day 25 (FIG. 1C) after surgery; rat with PBN treatment started on the day of surgery at day 11 (FIG. ID), day 23 (FIG. 1E) and day 39 (FIG. 1F) after surgery; rat with PBN treatment started 5 days before surgery at day 10 (FIG. 1G), day 34 (FIG. 1H) and day 39 (FIG. 1I) after surgery.

FIGS. 2A-D—T2-weighted images of 9L/LacZ gliomas. Control rat at day 6 (FIG. 2A) and day 17 (FIG. 2B) after surgery; rat with PBN treatment started 5 days before surgery at day 6 (FIG. 2C) and day 14 (FIG. 2D).

FIG. 3—Doubling time and tumor growth curves for C6 gliomas of control rat and rats with PBN treatment. Measurements on the day of surgery, 5 days before and 5 days after.

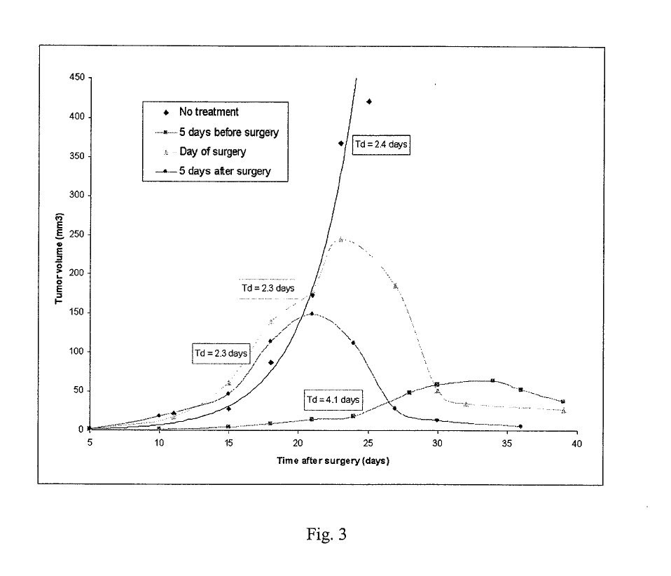

FIGS. 4A-E—Angiograms. Images obtained from a rat without a tumor (FIG. 4A); a rat with a C6 glioma without PBN treatment at day 23 (FIG. 4B); a rat with a C6 glioma with PBN treatment started on the day of surgery at day 30 (FIG. 4C); a rat with a C6 glioma with PBN treatment started 5 days before at day 30 (FIG. 4D); and a rat with a 9L/LacZ glioma without PBN treatment at day 13 (FIG. 4E).

FIGS. 5A-B—Example of voxels used for spectroscopy in the tumor region (FIG. 5B) and in the contralateral control side (FIG. 5A). The spectra obtained from these voxels depict the peaks of choline (1), creatine (2), NAA (3) and mobile lipids (methylene (4) and methyl lipid hydrogens (5)).

FIG. 6—Metabolite ratios. Data obtained by spectroscopy of a rat with a C6 glioma and PBN treatment started on the day of surgery.

FIG. 7—MRI tumor volumes. Rats implanted with C6 gliomas, and treated with PBN (75 mg/kg/day, drinking water) either 5 d before implantation of cells (red), on day of implantation (yellow) or 5 d after implantation (green), or no PBN treatment (blue). Top-left inset is iNOS Western blot of PBN and non-PBN brain tissues from control (con) and glioma (tum). MR images on right show a glioma without PBN treatment 25 d after implantation (top), and a PBN treated rat (5 d before implantation) (bottom).

FIGS. 8A-C—Histology (H&E) of control untreated or rat brain treated with PBN. Rat brains were either (FIG. 8A) normal, (FIG. 8B) C6 glioma injected or (FIG. 8C) PBN-treated 5 days after C6 glioma injection. Note that PBN-treated C6 glioma rat brain has neuronal cells that appear similar to control rat brain. Untreated C6 glioma depicts malignant cells that are atypical in appearance (grade II to III) with few mitotic and apoptotic figures being present.

FIG. 9—Anti-Glioma Therapy/PBN treatment of C6 gliomas. T2-weighted MR images of C6 gliomas (top series) without PBN treatment at days 7, 10 and 17 after intracerebral cell implantation; (middle series) continuous PBN treatment starting 5 days prior to cell implantation at days 7, 16, 21 and 27; and (bottom series) continuous PBN treatment starting 14 days after cell implantation (when the tumor is >50 mm³ at days 7, 16, 22 and 29. Method used is T2-weighted morphological MRI.

FIG. 10—Anti-Glioma Therapy / C6 glioma growth—PBN treatment. Tumor growth (tumor volumes in mm³) curves for C6 gliomas of non-PBN treated (closed diamonds), PBN treatment 5 days prior to cell implantation (closed squares), and PBN treatment 14 days after cell implantation (closed triangles). Method used is NIH ImageJ calculation of tumor volumes from T2-weighted MR image slices of entire C6 tumors without or with PBN treatment.

FIG. 11—Anti-Glioma Therapy/Percent Survival. Percent (%) survival data for rats not treated with PBN, treated with PBN 5 days prior to intracerebral cell implantation, and 14 days after cell implantation. Based on survival data obtained from FIG. 10.

FIG. 12—Anti-Glioma Therapy/F98 glioma growth—PBN treatment. Tumor growth (tumor volumes in mm³) curves for F98 gliomas (low grade) of non-PBN treated, and PBN treatment 5 days prior to cell implantation. Method used is NIH ImageJ calculation of tumor volumes from in vivo T2-weighted MR image slices of entire F98 tumors without or with PBN treatment.

FIG. 13—Anti-Glioma Therapy/C6 glioma angiogenesis—PBN treatment. Normalized (compared to contralateral control side of brain) blood volumes from (left hand panel) non-PBN and (right hand panel) PBN treated (5 days prior to C6 cell implantation) rat brains in glioma regions. Method used was quantitative analysis (using a Mathematica-based program developed in our laboratory) of arterial signal intensities from in vivo MR angiography data. Glioma region arterial blood volumes were compared to blood volumes in the contralateral control cerebral hemisphere.

FIG. 14—Anti-Glioma Therapy/Metabolite Spectroscopy. (Right hand panel) Image-guided MR spectroscopy in glioma tumor regions in non-PBN (C6 glioma) and PBN treated (C6 glioma PBN D-5 (PBN administed 5 days prior to cell implantation), and C6 glioma PBN D+15 (PBN administered 14-15 days after cell implantation) rats, compared to a normal brain region (Control). (Left hand panel) N-acetyl aspartate (NAA) to choline (Cho), and Lipids (at 1.3 ppm) to creatine (Cr) metabolite level ratios of a C6 glioma treated 5 days prior to cell implantation. Method used was image-guided MR spectroscopy in a volume of 125 mm³ or microliters in selected regions of control or tumor regions in rats in vivo. MR spectral peak areas were measured using the Bruker XWIN NMR peak intragration software package.

FIG. 15—T2-weighted MR images of C6 gliomas in rat brains. (Top row) non-40H-PBN-treated rats (5 days prior to C6 cell implantation) at 10, 15 and 18 days. (Middle row) 4OH-PBN-treated rats that had no treatment effect (n=3) at 7, 12 and 16 days. and (Bottom row) 4OH-PBN-treated rats that had an effect (n=2) at 7, 16 and 29 days following C6 glioma cell orthotopic implantation.

FIG. 16—Tumor growth curves of C6 gliomas in rats. Non-4OH-PBN-treated rats (blue) and 4OH-PBN-treated rats (as described in FIG. 15).

FIG. 17—Survival data for C6 gliomas in rats. Non-4OH-PBN-treated rats (blue) and 4OH-PBN-treated rats (as described in FIG. 15).

FIG. 18—Tumor morphology by MRI. Effects of 4-hydroxy PBN on tumor volume for five C6 glioma lines in Fischer 344 rats.

FIG. 19—Anti-glioma therapy. C6 glioma angiogenesis (ten lines) is monitored following treatment with 4-hydroxy PBN. Normalized arterial blood volumes of non-4OH-PBN and 4OH-PBN treated C6 gliomas.

FIG. 20—IGF-1 Induced HIF-1α Expression Inhibition by PBN. Inhibitory effects of PBN on IGF-1 induced HIF-1α expression in HCT-116 cells cultured at 20% O₂. The band densities of HIF-1α were normalized by the bands of CoCl₂ which was used as the positive control.

FIG. 21—IGF-1 Induced HIF-1α Expression Inhibition by PBN. Inhibitory effects of PBN on IGF-1 induced HIF-1α expression in Hepa 1-6 cells. The band densities of HIF-1α were normalized by the bands of CoCl₂ which was used as the positive control.

### DESCRIPTION OF ILLUSTRATIVE EMBODIMENTS

Gliomas are a complex family of brain tumors with different growth characteristics and involves different types of cells. Grading according to degree of malignancy was first proposed in 1949. In this classification, astrocytomas and glioblastomas represent different grades of malignancy of the same tumor. Grade I tumors, typically slow growing, are characterized by most cells having normal characteristics, and few mitotic features. Endothelial proliferation is absent. Grade II tumors, previously designated “astroblastomas,” are characterized by an increased number of cells with polymorphic nuclei in mitoses. There is no clear line of demarcation from normal tissue. Grade III tumors represent anaplastic astrocytomas, and Grade IV tumors represent the typical glioblastoma multiforme, characterized by cellular pleomorphism, vascular proliferation, mitoses, and multinucleated giant cells.

The current morphologically-based tumor classifications often mix cell lineage features with tumor growth characteristics. However, there are two general classifications—the anaplastic glioma strata and glioblastomas. The former is comprised of various gliomas including anaplastic astrocytomas, anaplastic oligoastrocytomas, anaplastic oligodendrogliomas, malignant glioma, anaplastic gliomas non-specified, and anaplastic ependymoma. Regardless of classification, there is an unfortunate lack of effective treatments for these aggressive and often fatal tumors.

1. The Present Invention

The present inventors have extended earlier work on the use of nitrone free radical trapping agent to treat various cancers including liver, stomach, colon, breast, pancreas, prostate, skin, head and neck, and blood tumors. However, the ability to treat gliomas, a notoriously difficult type of cancer to treat, was not addressed.

The present inventors decided to assess the effect of the anti-inflammatory phenyl N-tert-butyl nitrone (PBN) based on the presence of upregulated inflammatory genes identified using MRI techniques. As is shown below, this compound was able to control tumor development when provided to a subject either before, at the time of or after tumor implantation. Thus, it is now proposed to use PBN, and related nitrone free radical trapping agents, as therapeutic agents for gliomas.

2. Anaplastic Glioma Strata

A. Clinical Features

The anaplastic gliomas are intermediate grade infiltrative gliomas—classified between low (localized, slow growing) and glioblastoma multiforme (rapidly growing and highly invasive). Anaplastic astrocytomas (AA) are tumors that arise from brain cells called astrocytes and/or their precursors. Astrocytes are support cells of the central nervous system. The majority of astrocytic tumors in children are low-grade, whereas the majority in adults are high-grade. These tumors can occur anywhere in the brain and spinal cord.

Oligodendrogliomas are gliomas derived from oligodendrocytes and/or their precursors. Oligodendrocytes that have a role in the structure and function of myelinated neurons in the brain. Anaplastic oligodendroglioma (AO) are more aggressive than oligodendrogliomas, but are also more sensitive to chemotherapy than are anaplastic astrocytomas. A high rate of response to the use of PCV (procarbazine, CCNU, vincristine) chemotherapy has led to the common use of PCV chemotherapy prior to radiation therapy, following irradiation, and/or at tumor recurrence and progression. Another glioma appears as histologic mixture of both oligodendroglioma and astrocytoma tumor forms and is called oligoastrocytoma. While oligoastrocytoma can be low-grade, the majority of the mixed oligoastrocytomas are anaplastic oligoastrocytomas (AOA).

The last glioma subgroup are ependymomas. One subtype of malignant ependymomas is the anaplastic ependymoma (AE); these tumors arise from ependymal cells and/or their precursors that line the cerebrospinal fluid passageways, called ventricles. These tumors are classified as either supratentorial (in the top part of the head) or infratentorial (in the back of the head).

Clinical features and symptoms produced by gliomas depend on the location of the tumor and the age of the patient. The most common location for gliomas is in the cerebral hemispheres in adults and the cerebellum, brainstem, hypothalamus, and thalamus in children. Spinal cord gliomas are much less common then gliomas of the brain. Patients with these tumors have symptoms that vary depending on location in the brain or spinal cord. They can produce symptoms of headache, seizures, nausea and vomiting, limb weakness, unilateral sensory changes, personality change, and unsteadiness in walking.

B. Classifications

Anaplastic Astrocytoma. The histologic features of anaplastic astrocytomas are similar to those of low-grade astrocytomas but these features are more abundant and exaggerated. These tumors are WHO grade III (Kleihues et al., 1993; Kleihues and Cavenee, 2000). Cellularity is more increased, as are nuclear and cellular pleomorphism. These features may be extreme, with back-to-back cells and bizarre, hyperchromatic nuclei. Cytoplasm may be scanty, with nuclear lobation and enlargement indicating anaplasia. Mitotic activity is easily recognized in most anaplastic astrocytomas but inexplicably may be absent in areas with gemistocytes.

The range of anaplasia in this grade is broad, with some examples showing low cellularity and pleomorphism with a few mitotic figures and others being highly cellular and pleomorphic with frequent mitoses, lacking only the necrosis required for a histologic diagnosis of glioblastoma. For this reason, it is useful to have a more objective indicator of behavior, and some markers of cell proliferation have been used in an attempt to predict prognosis more accurately. The most used markers in this area have been antibodies to bromodeoxyuridine (BrdU) and Ki-67 (Davis et al., 1995). The cellular incorporation of BrdU is a specific marker of the DNA synthesis phase of the cell cycle, whereas the Ki-67 antibody labels an antigen that is present in all phases of the cell cycle except G₀. Both antibodies can be identified by immunohistochemical staining in paraffin-embedded tissue sections. As a generalization, higher labeling rates for anaplastic astrocytomas is associated with poor prognosis (Hoshino et al., 1993; Davis et al., 1995; Lamborn et al., 1999).

Glioblastoma multiforme. Glioblastoma, also known as glioblastoma multiforme, is the glioma with the highest grade of malignancy, WHO grade IV (Kleihues and Cavenee, 2000). It represents 15% to 23% of intracranial tumors and about 50%-60% of astrocytomas. Most examples are generally considered to arise from astrocytes because glial fibrillary acidic protein can be identified in the cell cytoplasm. Some examples, however, apparently arise from other glial lineages, such as oligodendrocytes. Glioblastoma is the most frequently occurring astrocytoma. Autopsy and serial biopsy studies have shown that some astrocytomas progress through the grades of malignancy with transformation from low-grade to anaplastic astrocytoma to glioblastoma (Muller et al., 1977). But, because some examples of glioblastoma appear to arise rapidly in otherwise normal patients and are recognized when they are small, it is thought that this variety of glioblastoma can also arise directly from malignant transformation of astrocyte precursor cells without passing through the lower grades of malignancy (Kleihues and Ohgaki, 1997; 1999).

Tumor necrosis is the characteristic gross feature that distinguishes glioblastoma from anaplastic astrocytoma (Nelson et al., 1983; Burger et al., 1985; 1991). Another microscopic feature that is distinctive and diagnostic is the presence of proliferative vascular changes within the tumor. These changes may occur in the endothelial cells (vascular endothelial hyperplasia or proliferation) or in the cells of the vessel wall itself (vascular mural cell proliferation). Both types of change are sometimes considered together as microvascular proliferation. Glioblastomas cellularity is usually extremely high. The individual cells may be small, with a high nuclear:cytoplasmic ratio, or very large and bizarre, with abundant eosinophilic cytoplasm. These same small cells may appear to condense in rows around areas of tumor necrosis, forming the characteristic pseudopalisades. Glioblastoma tumors have a propensity to infiltrate the brain extensively, spreading even to distant locations and giving the appearance of a multifocal glioma. Some examples are truly multifocal (i.e., arising in multiple simultaneous primary sites) while many of these multifocal tumors show a histologic connection when the whole brain is examined at autopsy.

Oligodendrogliomas. Like astrocytomas, oligodendrogliomas mimic the histology of their presumed cell of origin. They also arise primarily in the white matter but tend to infiltrate the cerebral cortex more than do astrocytomas of a similar grade of malignancy. Like astrocytomas, grading schemes of histologic malignancy have been used for oligodendrogliomas, but these correlate less well with prognosis than those used for astrocytomas (Burger et al., 1987; Bigner et al., 1998; Daumas-Duport et al., 1997). Many of the histologic features used to grade oligodendrogliomas are similar to those used for astrocytomas: cellularity, pleomorphism, mitotic activity, vascular changes, and necrosis. Lower-grade oligodendrogliomas may have microcysts. Oligodendrogliomas of all histologic grades tend to infiltrate the cortex readily and to form clusters of neoplastic cells in the subpial region, around neurons, and around blood vessels. In general, the cells of oligodendrogliomas have round, regular nuclei and distinct cytoplasmic borders with clearing of the cytoplasm. Another fairly distinctive and diagnostically helpful feature is the vascular pattern of oligodendrogliomas, referred to as “chicken-wire” vessels that can divide the tumor into discrete lobules. With increasing anaplasia, oligodendrogliomas can become highly cellular and pleomorphic, approaching an appearance of glioblastoma multiforme with the presence of necrosis. Although it is correct to classify these as anaplastic oligodendrogliomas, some would use the term glioblastoma once necrosis is identified in any high-grade glial neoplasm. One justification for separating anaplastic oliogdendrogliomas from astrocytic glioblastomas is the slightly better prognosis of the former, even in this highest grade of malignancy. Some authors have reported that a MIB-1 labeling index of >3%-5% predicts a worse prognosis in oligodendrogliomas (Heegard et al., 1995; Kros et al., 1996; Dehghani et al., 1998).

Oligoastrocytomas. Many, if not most, oligodendrogliomas occur with a regional or intimate cellular mixture of astrocytoma. For the diagnosis of mixed glioma, the proportion of each should be substantial, but authors have differing opinions with respect to exact numbers; usually a mixture with a range from 10% to 25% of the minor element is used to diagnose a mixed glioma. Oligoastrocytomas and anaplastic oligoastrocytomas correspond to WHO grade II or grade III, respectively (Kleihues and Cavenee, 2000). Histologic features of anaplasia may be present in either component and will affect the prognosis adversely. Such features include marked cellular pleomorphism, high cellularity, and a high mitotic rate. Microvascular proliferation and necrosis may also be seen. Prognosis and response to therapy have not been shown to depend on the proportion of the oligodendroglial versus the astrocytic component (Shaw et al., 1994), although paradoxically, the BrdU LI of the oligodendroglial component is more predictive for survival than the astrocytic component (Wacker et al., 1994) and far advanced tumor progressions are dominated by the astrocytic component.

3. Nitrone Free Radical Trapping (NFRT) Agents

The compound phenyl N-tert-butyl nitrone (PBN) was first synthesized in the 1950's, but in 1968 it was discovered to be very useful to trap and stabilize free radicals in chemical reactions and hence it was termed a spin-trap (Janzen, 1971). Although PBN is the prototype spin-trap, several other nitrones have been synthesized and found useful to trap and characterize free radicals in chemical reactions. These spin traps were used in chemical reactions first, but in the mid-1970's they began to be used to trap free radicals in biochemical and biological systems (Floyd et al., 1977; Poyer et al., 1978). Pharmacokinetic studies have shown that PBN is readily and rapidly distributed almost equally to all tissues, has a half-life in rats of about 132 minutes and is eliminated mostly in the urine. Relatively few metabolism studies have been done, but it is known that some ring hydroxylation (primarily in the para position) of the compound occurs in the liver.

Novelli first showed that PBN could be used to protect experimental animals from septic shock (Novelli et al., 1986), and indeed this was later confirmed by other groups (Pogrebniak et al., 1992). The use of PBN and derivations as pharmacological agents began after discoveries in 1988 that showed that PBN had neuroprotective activity in experimental brain stroke models (Floyd, 1990; Floyd et al., 1996; Carney et al., 1991). These results were repeated and extended, (see Clough-Helfman et al., 1991; Cao et al., 1994; Folbergrova et al., 1995; Pahlmark et al., 1996). Others inventors have summarized the extensive neuroprotective pharmacological research effort on PBN and derivatives (Floyd, 1997; Hensley et al., 1996). In addition to neurodegenerative diseases, PBN has been shown to protect in other pathological conditions where ROS-mediated processes are involved, including diabetes and many other conditions. The mechanistic basis of why PBN and some of its derivatives are so neuroprotective in experimental stroke and several other neurodegenerative models has not been completely elucidated yet. However, it is clear that its action cannot simply be explained by its ability to trap free radicals.

Research on the mechanistic basis of PBN's action now shows that it is acting by suppressing gene induction (Floyd, 1997; Hensley et al., 1996; Miyajima et al., 1995; Tabatabaie et al., 1996; Hensley et al., 1997; and Kotake, 1999), most likely by acting on oxidation-sensitive signal transduction processes (Sang et al., 1999). In fact, PBN seems to be acting by suppressing signal transduction enhanced ROS formation by mitochondria (Hensley et al., 1998). These findings and ideas have arisen from the study of neurodegenerative processes.

Other inroads into defining the mechanism of PBN's anti-cancer function have recently been made. Hypoxia Inducing Factor α (HIF-1α) is important in biomedical problems where anoxia/reoxygeneration events occur including heart attacks, stroke, cancer development, kidney malfunction, circulatory complications of diabetes and many other problems that are generally associated with aging. Tissue anoxia activates a massive change in gene induction leading to activation of over 100 genes mostly beneficial in the adaption of the tissue to the lack of oxygen. Long term anoxia can lead to serious complications including death of the tissue. The transcription factor which can mediate the induction of these genes is known as HIF-1α. The mechanism of the action of HIF-1α and its regulation is becoming increasingly understood.

Oxygen is necessary for aerobic life. Hypoxia causes a major adaptive response. Suites of genes are induced that code for enzymes to enhance glycolytic metabolism and other proteins (VEGF, etc.) to enhance angiogenesis for the development of new blood vessels for the delivery of oxygen, for example. In order for cancerous tissue to develop and grow there must be an adequate supply of oxygen. A tumor larger than 2 mm diameter becomes hypoxic at the core thus leading to cell death unless a supply of oxygen is established. Therefore angiogenesis must occur in order to achieve further growth. Hypoxia brings about the activation of the HIF-1 transcription factor. HIF-1 is a transcription factor capable of bringing about the induction of suites of genes, at least 100, as an adaptive response. VEGF is one such gene product that aids in triggering angiogenesis necessary for further tumor growth. Even though HIF-1 is capable of acting as a transcription factor for many genes there must be many other factors that also exert control over the orchestration of various suites of genes in the response to hypoxia. This seems especially true for the particular genes we are focused on, i.e., iNOS and VEGF. The inventors have found studying certain cell systems that even if hypoxia induces VEGF 10-fold this is not necessarily the case for iNOS under the same conditions. This clearly implicates several other factors other than the level of active HIF-1 controls the magnitude and timing of the induction of specific genes transactivated by HIF-1. Based on the background literature it is clear that depending on the cell system, various signal transduction process exert control of iNOS expression. It is not clear how various cell signaling processes interface and coordinate with HIF-1 in the control of iNOS expression.

It has been over 10 years since HIF was discovered (Semenza and Wang, 1992; Wang and Semenza, 1993a; Semenza et al., 1994) and first characterized (Wang and Semenza,1993b; Wang and Semenza, 1995; Wang et al., 1995). Since then an enormous amount of research has been done and much learned regarding the physiological and developmental role of HIF and especially its role in cancer development. Briefly in general the cellular response to hypoxia is mediated through HIF-1 which acts as a transcription factor (Welsh et al., 2004). HIF-1 consists of a heterodimer of HIF-1α and HIF-1β. HIF-1β is also known as the aryl hydrocarbon receptor protein. HIF-1α and HIF-1β associate in the cytosol before migrating to the nucleus where they bind to hypoxic-regulated elements (HRE) consisting of -CACGTG- in DNA sequences in both the 3′ and 5′ regions of hypoxia-regulated genes. HIF-1β is constitutively expressed and the levels are not altered by hypoxia. HIF-1α is also constitutively expressed but under aerobic conditions it is rapidly degraded by ubiquitin-26S proteosomes so that very low levels of HIF-1α are present under normal aerobic conditions. However, when hypoxia occurs HIF-1α is not degraded so its levels increase causing it to be present at much higher levels and thus under these conditions HIF-1 transactivates hypoxia inducible genes.

There are a large number of genes induced by hypoxia; in fact over 100 have been documented (Vaupel, 2004). The most pertinent genes in regard to cancer development induced by hypoxia are those involved in glycolytic metabolism such as Glut 1 and several of the glycolytic enzymes, those that promote erythropiesis such as erythropoietin, genes that protect against apoptosis, genes that promote angiogenesis such as VEGF and iNOS. The importance of HIF-1α in cancer development has been reviewed recently (Vaupel, 2004; Kaelin, Jr., 2005; Maxwell, 2005). Much recent research has been devoted to discovering novel therapeutics based on the emerging understanding of the action of HIF-1α in cancer development (Kaelin, Jr., 2005; Maxwell, 2005). HIF-1α is more prominently expressed in hypoxic regions of tumors because it is stable at lower oxygen regions in contrast to regions of higher oxygen tension where it is degraded by proteosomes an end result of HIF-1α being oxidized at a conserved asparagine in the N-terminus and two proline residues in the C-terminus (Kaelin, Jr., 2005; Maxwell, 2005; Dann and Bruick, 2005). These oxidations are carried out by dioxygenase enzymes dependent upon 2-oxoglutarate as substrate which is oxidized to succinate. Oxidation of HIF-1α is also dependent upon ascorbate and Fe as a cofactor (Kaelin, Jr., 2005; Maxwell, 2005; Dann and Bruick, 2005). The oxidized HIF-1α is then polyubiquitinated and degraded by proteosomes.

Phosphorylation of HIF-1α has been shown to occur in several systems (Richard et al., 1999; Hofer et al., 2001; Nikinmaa et al., 2004; Gradin et al., 2002; Kwon et al., 2005). Even though this has not been studied extensively it is clear that phosphorylation of HIF-1α stabilizes this protein and it is active as a transcription factor (Minet et al., 2001). It has been demonstrated that ERK, p41 as well as p44, are capable of directly phosphorylating HIF-1α however neither p38MAPK nor JNK were able to do so in the system used (Richard et al., 1999). It was noted that the transcription activity of HIF-1α was enhanced by phosphorylation (Richard et al., 1999), however they were unable to definitely show which amino acids were phosphorylated in HIF-1α. Gradin et al. later demonstrated that threonine 844 was phosphorylated and this was necessary for transcription activity (Gradin et al., 2002). Utilizing MiaPaCa-2 pancreatic cancer cells where low glucose and ischemia were imposed thereby enhancing the presence of reactive oxygen species and causing activation of the ASK-1 pathway, Kwon demonstrated that activated P₃₈ (in fact all four forms, i.e., α, β, δ and γ) were capable of phosphorylating HIF-1α in vitro (Kwon et al., 2005). P₃₈ proteins from quiescent cells did not phosphorylate HIF-1α. Phosphorylation of HIF-1α stabilized this transcription factor because it was prevented from binding to the von Hippel-Lindau (vHL) tumor suppressor protein, a necessary key event in the ubiquitination and proteosomal degradation of HIF-1α.

It has been demonstrated that several signal transduction pathways are involved in HIF-1 induction even under normoxia conditions (Bardos and Ashcroft, 2005). IGF-1 has been demonstrated to induce HIF-1α in several cell lines under normoxia conditions (Bardos and Ashcroft, 2005; Chau et al., 2005; Fukuda et al., 2002; Zundel et al., 2000; Dimova et al., 2005). IGF-1 induction of HIF-1α under normoxia in an osteosarcoma cell line has been shown useful to screen for small molecule inhibitors of HIF-1α induction (Chau et al., 2005). Two effective novel inhibitors, NSC-134754 and NSC-643735, were found in the National Cancer Institutes Diversity set of 2000 compounds. NSC-134754 is a semisynthetic analogue of the natural alkaloid emetine and NSC-643735 is structural analogue of actinomycin D. Inhibition of HIF-1α induction by small molecule inhibitors in IGF-1 mediated processes were compared to HIF-1α induction under hypoxia as well as that by dieferoxamine mesylate (Dfx) which chelates the Fe in the prolyl hydroxylase enzyme thus preventing it from acting upon HIF-1α thereby mediating its targeting for proteolytic decomposition (Chau et al., 2005). Results obtained were similar in all three methods of HIF-1α induction. It is interesting to note that after the induction by hypoxia, HIF-1α was rapidly decreased (approximately 80%) by 5 min of incubation of the cells in normoxia conditions (Chau et al., 2005). Utilizing HCT116 colon cancer cells and IGF-1 induction Semenza's group demonstrated that HIF-1α was induced via either the IGF-1/MEK/ERK route or the IGF-1/P13K/AKT/FRAP route (Fukuda et al., 2002). The importance of the AKT route of HIF-1α induction has been demonstrated in several glioblastoma cell lines when PTEN was inactivated (Zundel et al., 2000).

Several reports clearly show that higher levels of thioredoxin-1 (Trx-1) enhanced the stability and hence activity of HIF-1α (Welsh et al., 2002; Welsh et al., 2003; Kim et al., 2005; Csiki et al., 2006). First, overexpression of Trx-1 in cancer cells caused enhanced HIF-1α transcription of gene products VEGF and iNOS and enhanced tumor growth in mice xenograph studies (Welsh et al., 2002). Second, pharmacologic treatment with PX-12 (an irreversible Trx-1 inhibitor) or pleurotin (a thioredoxin reductase inhibitor) caused decreased HIF-1α gene transcription products (VEGF and iNOS) and caused decreased tumor growth (33427}. Third, treatment of tumor cells with antisense Trx-1 caused decreased angiogenesis as expected by decreased HIF-1 activity (Kim et al, 2005). Fourth, overexpression of Trx-1 in non-small cell lung cancer caused the enhanced presence of HIF-1α and its gene products including COX-2 (Csiki et al., 2006). Welsh et al. using Trx-1 transfected MCF-7 human breast and HT-29 human colon carcinoma cells as well as WEHI7.2 mouse lymphoma cells demonstrated that a significantly increased amount of HIF-1α protein and an increase in proteins of hypoxia-responsive genes (VEGF and iNOS) when compared to non-transfected cells (Welsh et al., 2002). Transfection with redox-inactive Trx-1 markedly decreased HIF-1α protein and decreased VEGF expression, therefore indicating the redox activity of Trx-1 was of importance in the enhancement of the presence of HIF-1α and activity. Increased tumor growth was noted when the Trx-1 transfected WEHI7.2 cells were injected into scid mice as compared to that noted when the non-transfected cells were used. Increased angiogenesis was noted in the tumors caused by Trx-1 transfected cells. The mechanistic basis of how Trx enhances HIF-1 is not known but it most likely related to redox regulated molecular processes. Very early it was discovered that H₂O₂ treatment of cell extracts prevented HIF-1 from binding to DNA. This was overcome by treating the extract with Trx or with agents that reduces oxidized sulfhydryl groups (Huang et al., 1996). This effect of Trx was presumed to be done to Trx reduction of oxidized cysteines on the proteins that directly interacted with DNA. Welsh et al. interpreted the action of Trx in their system by the possible reduction of Ref-1 that enhances the binding of CBP/p300 to HIF-1α.

Heat shock protein 90 (HSP90) is known to regulate a pathway of degradation of HIF-1α (Martinez-Ruiz et al., 2005; Kubo et al., 2004; Minet et al., 1999; Ibrahim et al., 2005; Mabjeesh et al., 2002). It has been known for some time that HSP90, a chaperone protein, binds with HIF-1α (Gradin et al, 1996). Therefore agents that inhibit the action of HSP90, such as geldanamycin (Kubo et al., 2004; Mabjeesh et al., 2002; Isaacs et al., 2002) as well as the environmental agent bisphenol A (Kubo et al., 2004), enhances the rapid degradation of HIF-1α. It is known that HSP90 is S-nitrosylated by incubating endothelial cells with S-nitroso-L-cysteine (CysNO) (Martinez-Ruiz et al., 2005). A cysteine on the C-chain is S-nitrosylated which prevents the normal binding of HSP90 to eNOS (Martinez-Ruiz et al., 2005). S-nitrosylation also prevents HSP90 from hydrolyzing ATP.

As discussed above, the invnetors have found that PBN is potently effective in preventing the development of gliomas and glioblastomas in rat models. The mechanistic studies conducted by the inventors on the anti-cancer activity of PBN implicate that its activity is strongly coupled to its ability to suppress the induction of inducible nitric oxide synthase (iNOS) therefore preventing higher levels of nitric oxide (NO) formation in the target tissue (Floyd, 2006). Their interpretation of the many studies in several human tumors as well as animal models of cancers clearly demonstrate that NO produced by iNOS at modest levels is a very important agent that enhances the continued growth and development of tumors. PBN acts to inhibit tumor growth and development by preventing NO formation, specifically by inhibiting the expression of iNOS. These background studies provide a strong scientific mechanistic basis to explain why iNOS/NO is important in tumor development and explains why specific nitrones are expected to be effective anti-cancer agents with significant commercial potential. Recently, the inventors have shown that PBN inhibits c-MET, a tyrosine kinase inhibitor, associated with invasive cell growth, and very recent results by the inventors have shown that PBN inhibits the induction of HIF-1α in several cancer cells. HIF-1α is a major transactivation factor which is central to the induction of VEGF as well as iNOS and many other genes very important in the development of cancer.

U.S. Pat. No. 5,569,902 (incorporated herein by reference) describes the use of NFRT agents for the treatment of cancer. Specifically, PBN and related compounds are described as being useful in the preparation of an anti-carcinogenic diet and the preparation of such supplemented diets. Those subjects most likely to beneficially receive the nitrones would include: 1) those having had pretumor tests indicating a high probability of the presence of tumors, 2) those exposed to very potent carcinogenic environments and their probability of tumor progression is high, and 3) to those whose genetic predisposition makes their likelihood of tumor development high.

PBN and derivatives are contemplated, including hydroxy derivatives, especially 2-, 3- or 4-hydroxy PBN and mono-, di- and trihydroxy tert-butyl nitrone; esters, especially esters which release 2-, 3, or 4-hydroxyphenyl t-butyl nitrone such as the acetoxy derivative, 2-, 3-, or 4-carboxyphenyl t-butyl nitrone, such as the ethyl derivative, or phenyl hydroxybutyl nitrone, such as the acetoxy derivative; alkoxyl derivatives, especially alkoxyl derivatives which release 2-, or 4-hydroxyphenyl t-butyl nitrone, such as the methyl derivative; and acetamide derivatives, especially acetamide derivatives which release 2-, or 4 aminophenyl t-butyl nitrone, such as the acetyl derivative; diphenyl nitrone (PPN) and the analogous diphenyl nitrone derivatives.

The general formula for PBN and useful derivatives thereof is: 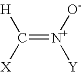
wherein:
X is phenyl or 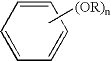
wherein R is H, 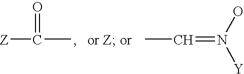
and n is a whole integer from 1 to 5; or 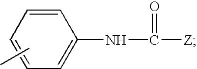
Y is a tert-butyl group that can be hydroxylated or acetylated on one or more positions; phenyl; or 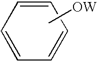
wherein W is 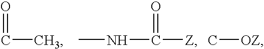
or Z; and Z is a C₁ to C₅ straight or branched alkyl group.

Other spin-trapping agents can also be used, such as 5,5-dimethyl pyrroline N-oxide (DMPO) or α-(4-pyridyl 1-oxide)-N-tertbutylnitrone (POBN), and spin-trapping derivatives thereof. Derivatives are made using standard techniques, for example, for substitution of the methyl groups. The general formula for DMPO is:

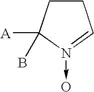

wherein A and B are independently CH₃, CH₂ OH, CH.sub.2 OW, or

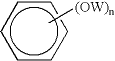

n is an integer from 1 to 5 wherein W is

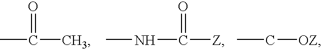

or Z; and Z is a C₁ to C₅ straight or branched alkyl group. The general formula for POBN is:

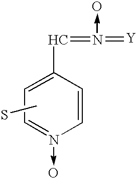

wherein Y is a tert-butyl group that can be hydroxylated or. acetylated on one or more positions; phenyl; or

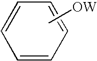

wherein W is

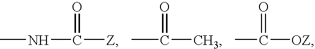

or Z; and S═H, (OR)ₙ, wherein R is H,

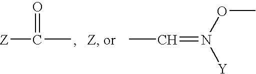

n is a whole number from 1 to 4, or

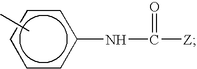

Z is a C₁ to C₅ straight or branched alkyl group.

Similarly, the nitrone free radical trapping agents of the present invention are characterized by phenyl, alkyl-substituted nitrones. The basic core structure of the phenyl nitrone is so simple that there are only a few thousand practically synthesized derivatives. A particular nitrone is an aryl N-alkyl nitrone. The alkyl may be tertiary (tert) butyl, although other alkyls, cycloalkyls and the like may be used. Specific aryls are phenyl, 3-hydroxyphenyl and 4-hydroxyphenyl and the like. Specific compounds include 3-hydroxyphenyl N-tert-butylnitrone, 2-hydroxyphenyl N-tert-butylnitrone, 2-sulfoxyphenyl N-tert-butylnitrone and 4-hydroxyphenyl N-tert-butylnitrone. Also encompassed are modified butyl nitrone compounds such as 5,5-dimethyl-1-pyrroline-N-oxide and alpha(4-pyridyl- 1 -oxide)-N-tert-butyl nitrone.

4. Combination Treatments

In one embodiment, the nitrone free radical trapping agents of the present invention may be used in conjunction with another glioma therapy, such as radiation, PCV, DFMO, CCNU or BCNU. These compositions would be provided in a combined amount effective to kill or inhibit proliferation of the cell. This process may involve contacting the cells with the agents at the same time. This may be achieved by contacting the cell with a single composition or pharmacological formulation that includes both agents, or by contacting the cell with two distinct compositions or formulations, at the same time, wherein one composition includes the NFRT agent and the other includes the second agent.

Alternatively, the NFRT agent therapy may precede or follow the other agent treatment by intervals ranging from minutes to weeks. In embodiments where the other agent and NFRT agent are applied separately to the cell, tissue or organism, one would generally ensure that a significant period of time did not expire between the time of each delivery, such that the agents would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Multiple administrations of each agent are contemplated. For example, where the NFRT agent is “A” and the secondary agent is “B”, the following are contemplated:

| | | | | |
|---|---|---|---|---|
| A/B/A | B/A/B B/B/A | A/A/B A/B/B | B/A/A A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Patients will be evaluated for neurological changes considered to be independent of tumor and graded using NCI Common Toxicity Criteria (neurotoxicity). Aside from baseline audiometric testing, repeat audiometric testing for ototoxicity is performed at the physician's discretion for patients who had evidence of hearing loss or progression of hearing loss by neurological examination. In addition, blood counts are performed biweekly, and serum creatinine, alkaline phosphatase, bilirubin and alanine amino-transferase tests are performed before each cycle. Doses may be modified during the course of treatment, primarily based on neutrophil and platelet counts (vincristine, lomustine and matulane) or ototoxicity (DFMO). Occasionally, DFMO dose reductions are required for diarrhea.

A. PCV

PCV is a drug combination therapy employing three different agents—a hydrazine derivative, matulane, a nitrosourea, lomustine, and a tubulin interactive agent, vincristine. It has been used in a number of clinical trials, most notably by the inventor in assessing its effect on high-grade glioma and medulloblastoma tumors. The major side-effect observed with PCV was dose-limiting myelotoxicity. Each of the components of PCV is described below.

It should be noted that the present invention could include the use of BCNU rather than of CCNU (lomustine) since both are nitrosoureas. It also is contemplated that one could use CCNU and procarbazine or BCNU and procarbazine, without vincristine, since vincristine is usually considered to be the least active of the drugs in the PCV combination.

Both hydrazines and nitrosoureas are alkylating agents. As a group, alkylating agents form covalent chemical adducts with cellular DNA, RNA and protein molecules and with smaller amino acids, glutathione and similar chemicals. Generally, these alkylating agents react with a nucleophilic atom in a cellular constituent, such as an amino, carboxyl, phosphate, sulfhydryl group in nucleic acids, proteins, amino acids, or glutathione. The mechanism and the role of these alkylating agents in cancer therapy is not well understood. In addition to hydrazine and nitrosoureas, aklyating agents include: triazenes such as dacarabzine and temozolomide, nitrogen mustards such as chlorambucil, cyclophosphamide, isofamide, mechlorethamine, melphalan, uracil mustard; aziridine such as thiotepa; methanesulphonate esters such as busulfan; platinum complexes such as cisplatin, carboplatin; bioreductive alkylators, such as mitomycin and and altretemine. Any of these compounds may be used together or individually, in combination with the compounds of the present invention.

### i. HYDRAZINE AND TRAIZENE DERIVATIVES

Hydrazine and triazene deriviatives are similar to nitrosoureas in that they decompose spontaneously or are metabolized to produce alkyl carbonium ions, which alkylate DNA. This class of compounds includes matulane, dacarbazine and temozolomide.

The active ingredient in matulane is Procarbazine Hydrochloride (N-isopropl-alpha-(2-methylhydrazino)-p-toluamide monohydrochloride). It is available from Roche Laboratories, Inc. It was approved in 1969 for treatment of Hodgkins' Disease. The typical form is an oral capsule that contains 50 mg procarbazine as the hydrochloride. Dosages vary depending upon whether procarbazine is being used as a combination drug with other anticancer drugs or as a single therapeutic agent. A suggested guideline per the PDR for single agent use is 100 mg two times daily for 14 days.

The exact mode of actions of matulane is not clear. There is some evidence that the drug acts by inhibition of protein, RNA and DNA synthesis. It is primarily metabolized in the liver and kidneys and appears to be auto-oxidized to the azo derivative with the release of hydrogen peroxide. The azo derivative isomerizes to the hydrazone and, following hydrolysis, splits into a benzylaldehyde derivative and methylhydrazine. The methylhydrazine is further degraded to CO₂ and CH₄, and possibly hydrazine, whereas the aldehyde is oxidized to acid which is excreted in the urine.

Matulane exhibits monamine oxidase inhibitory activity (MAOI), so a diet that restricts foods which contain high tyramine content should be followed. Drugs to be avoided during therapy include antihistamines, sympathomimetics, barbiturates, narcotics, hypotensive agents or phenothiazines, and ethyl alcohol. Some foods are also to be avoided during procarbazine such as naturally aged cheeses, chocolates, nuts, and bananas as they could theoretically lead to a hypertensive complication in some patients. Also, unacceptable toxicity may occur if matulane is used in patients with impairment of renal and/or hepatic function. Treatment may be curtailed in the event of central nervous system signs or symptoms such as paresthesias, neuropathies or confusion; neutropenia (absolute neutrophil count under 1500/ul), thrombocytopenia (platelets under 100,000/ul), hypersensitivity reaction, ulceraction or persistent spot of soreness around the oral cavity, diarrhea or loose stools, hemorrhage or bleeding tendencies.

Adverse but expected reactions include leukopenia, neutropenia, anemia, and thrombocytopenia. Commonly reported acute side effects are nausea and vomiting during or shortly after dose administration.

### ii. NITROSOUREAS

Nitrosoureas represent a group of therapeutic alklyating agents. This class of compounds includes lomustine, carmustine, semustine, steptozocin, and nimustine.

### (a) Lomustine

Lomustine is a synthetic akylating agent, also known as CCNU, with the chemical name of 1-(2-chloroethyl)-3-cyclohexyl-1-nitrosourea. It was approved in 1977 for treatment of brain tumors and Hodgkin's Disease. It is available from Bristol Myers Squibb as oral capsule, available in 10 mg, 40 mg and 100 mg forms. Dosages may vary depending upon whether lomustine is being used as a single agent or in a combination in addition to other chemotherapeutic agents. As a single agent in previously untreated patients, the recommended dosages per the PDR is 130 mg as a single oral dose every 6 weeks. Lomustine crosses the blood brain barrier.

It is believed that CCNU alkylates DNA and RNA. It is cross-resistant with other nitrosoureas and some but not all alkylating agents. It may also inhibit several key enzymatic processes by carbamoylation of amino acids in proteins.

The most common and severe toxic side effects are bone marrow suppression leading to thrombocytopenia and leukopenia, which may contribute to bleeding and infections. Bone marrow toxicity is cumulative and thus dosage adjustments must be considered on the basis of the nadir blood counts from prior doses.

### (b) Carmustine

Carmustine, also known as BCNU, with the chemical name of N,N′-Bis(2-chloroethyl)-N-nitrosurea, is a nitrosurea alkylating agent approved by the FDA in 1977. Carmustine has ben used for many years for treatment of primary brain tumors and is used for the treatment of gliomas. Carmustine is available from Bristol Meyers Squibb in packages containing vials of 10 mg carmustine and 3 ml sterile diluent for delivered by i.v. injection. As a single agent carmustine is administered at about 150-200 mg/m² every 6 weeks. In combination regimens, carmustine may be given in does similar to those of lomustine. An alternative mode of delivery is by wafers implanted directly into the tumor site (Gliadel® Wafer).

Potential side effects include bone marrow suppression, anemia, diarrhea, low white blood cell and platelet counts, pulmonary toxicity and swallowing difficulties.

### iii. TUBULIN INTERACTIVE AGENTS

Tubulin interactive agents interfere with cell division by binding to specific sites on Tubulin, a protein that polymerizes to form cellular microtubules. Microtubules are critical cell structure units. When the interactive agents bind on the protein, the cell cannot properly form microtubules. Tubulin interactive agents include vincristine and vinblastine, both alkaloids and the taxanes, such as paclitaxel and docetaxel.

Vincristine, is available as Oncovin™ from Eli Lilly & Company and as Vincristine Sulfate from Faulding. Also called vincaleukoblastine, a 22-oxo-, sulfate (1:1) (salt), the salt of an alkaloid obtained from a common flowering herb, the periwinkle plant. It is delivered by intravenous injection. It was approved in 1963 on label for Ewing's Sarcoma, rhabdomyosarcoma, Wilm's Tumor, neuroblastoma, Hodgkin's Disease and leukemia.

The mechanism of action remains under investigation; however, there is an indication that inhibition of microtobule formation in the mitotic spindle, resulting in an arrest of dividing cells at the metaphase state, is involved. The liver is the major excretory organ. Most of an intravenous dose of Vincristine is excreted into the bile after rapid tissue binding. Vincristine does not appear to cross the blood brain barrier.

Vincristine has been reported to reduce blood levels of antiseizure medications and to increase seizure activity. The most common adverse reaction is hair loss. Leukopenia, neuritic pain and constipation occur, but usually for less than 7 days.

B. DFMO

Numerous highly proliferative types of cancer are associated with increased levels of the polyamines putrescine, spermidine, and spermine in tumor tissue and blood and urine of mammals with cancer. Studies have shown that this can be related to increased polyamine synthesis by the rate-limiting enzyme, omithine decarboxylase (ODC). The pathway for polyamine synthesis begins with L-omithine. This natural amino acid, although not normally incorporated into proteins, is part of the urea cycle which metabolizes arginine to ornithine and urea. Omithine is converted by omithine decarboxylase (ODC) to putrescine and CO₂ and is considered to be the rate-limiting step in the production of polyamines. With the addition of propylamine donated from S-adenosylmethionine, putrescine is converted to spermidine. Spermidine is then converted to spermine by spermine synthetase, again in association with the decarboxylation of S-adenosylmethionine. Putrescine, spermidine and spermine represent the three major polyamines in mammalian tissues. Polyamines are found in animal tissues and microorganisms and are known to play an important role in cell growth and proliferation. Although the exact mechanism of the role of the polyamines in cell growth and proliferation is not known, it appears that the polyamines may facilitate macromolecular processes such as DNA, RNA, or protein synthesis. Polyamine levels are known to be high in the testes, ventral prostate, and thymus, in psoriatic skin lesions, and in other cells undergoing rapid growth processes.

It also is well known that the rapid proliferation of tumor tissue is marked by an abnormal elevation of polyamine levels. Hence, the polyamines also may play an important role in the maintenance of tumor growth. Thus, ODC inhibitors, such as DFMO, may exert their therapeutic effect by blocking the formation of the polyamines and thereby slowing, interrupting, or arresting the proliferation and metastases of the tumor tissue.

DFMO (alpha-difluoromethylornithine, eflomithine, Omidyl®) is a structural analog of the amino acid L-omithine and has a chemical formula C₆H₁₂N₂O₂F₂. DFMO can be employed in the methods of the invention as a racemic (50/50) mixture of D- and L-enantiomers, or as a mixture of D- and L-isomers where the D-isomer is enriched relative to the L-isomer, for example, 70%, 80%, 90% or more by weight of the D-isomer relative to the L-isomer. The DFMO employed may also be substantially free of the L-enantiomer.

The dose limiting toxic effect of DFMO is thrombocytopenia (abnormally few platelets in the blood), which occurs in about fifty percent of patients, leukopenia (abnormally few leukocytes), or anemia. This toxic effect is relatively harmless and reversible and cease upon withdrawal of the drug.

The effect of an ODC inhibitor for the control of the growth rate of rapidly proliferating tumor tissue has been assessed in standard animal tumor models. For example, the anti-tumor effect of DFMO has been demonstrated in the following animal tumor models: L1210 leukemia in mice, EMT6 tumor in Balb/C mice, 7,12-dimethylbenzanthracene-induced (DMBA-induced) mammary tumor in rats, and DFMO Morris 7288C or 5123 hepatoma in Buffalo rats. In addition, the anti-tumor effect of DFMO in combination with various cytotoxic agents has been demonstrated as follows: (a) in combination with vindesine or adriamycin in L1210 leukemia in mice, in Morris 7288C hepatoma in Buffalo rats, and in EMT6 tumor in mice, (b) in combination with cytosine arabinoside in L1210 leukemia in mice, (c) in combination with methotrexate in L1210 leukemia in mice, (d) in combination with cyclophosphamide in EMT6 tumor in mice and in DMBA-induced tumor in mice, (e) in combination with BCNU in mouse glioma 26 brain tumor, and (f) in combination with MGBG in L1210 leukemia in mice, in Morris 7288C hepatoma in Buffalo rats, in P388 lymphocytic leukemia in mice, and in S-180 sarcoma in mice.

Although DFMO can effectively block tumor putrescine biosynthesis, the resultant antitumor effect is cytostasis, not cytotoxicity. For example, DFMO reduces the growth rate of an MCA sarcoma, but does not produce tumor regression. This finding is consistent with reports of other investigators who showed that DFMO is a cytostatic agent. However, studies indicate that a significant role may exist for DFMO agents, permitting the future development of combination chemotherapeutic regimens which incorporate DFMO.

The initial promise of DFMO as a therapeutic ODC inhibitor for use in the treatment of various neoplasias has dimmed somewhat because, although DFMO does, in fact, irreversibly inhibit ODC activity, cells treated in vivo with DFMO significantly increase their uptake of exogenous putrescine as described in U.S. Pat. No. 4,925,835. The intercellular transport mechanisms of the cell do an “end run” around the DFMO-impaired ODC activity by importing putrescine from the extracellular milieu. Therefore, DFMO's effect in vivo is far poorer than in vitro. So, while DFMO treatment effectively inhibits intracellular putrescine neogenesis, it also results in increased uptake of extracellular putrescine, thereby offsetting its ODC inhibitory effect.

This problem is compounded by the fact that putrescine is present in many common foods, such as grapefruit juice, which contains approximately 400 ppm putrescine. This makes it virtually impossible to provide a patient a nutritionally sufficient diet which is free of putrescine. Therefore, DFMO-treated cells are capable of importing sufficient amounts of extracellular putrescine to support cell division.

Strategies to make DFMO more acceptable to human patients are described in U.S. Pat. No. 4,859,452 (incorporated by reference). Formulations of DFMO are described which include essential amino acids in combination with either arginine or omithine to help reduce DFMO-induced toxicities.

C. Radiation

Factors that cause DNA damage and have been used extensively for cancer therapy and include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells. The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

D. Surgery

Approximately 60% of persons with cancer will undergo surgery of some type, which includes preventative, diagnostic or staging, curative and palliative surgery. Curative surgery as a cancer treatment may be used in conjunction with other therapies, such as the treatment of the present invention, chemotherapy, radiotherapy, hormonal therapy, gene therapy, immunotherapy and/or alternative therapies. Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

5. Pharmaceutical Formulations

The present invention discloses numerous compositions, which in certain aspects of the invention, are administered to animals. For example, the instant invention discloses NFRT agents, as well as various secondary chemotherapeutic agents. Where clinical applications are contemplated, it will be necessary to prepare pharmaceutical compositions of these compounds and compositions in a form appropriate for the intended application. Generally, this will entail preparing compositions that are essentially free of pyrogens, as well as other impurities that could be harmful to humans or animals.

One will generally desire to employ appropriate salts and buffers to render agents suitable for introduction into a patient. Aqueous compositions of the present invention comprise an effective amount of the agent, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients, such as other anti-cancer agents, can also be incorporated into the compositions.

Solutions of the active ingredients as free base or pharmacologically acceptable salts can be prepared in water suitably mixed with surfactant, such as hydroxypropylcellulose. Dispersions also can be prepared in glycerol, liquid polyethylene glycols, mixtures thereof and in oils. Under ordinary conditions of storage and use, these preparations contain a preservative to prevent growth of microorganisms. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

An effective amount of the agents is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject, and the protection desired. Precise amounts of the therapeutic composition also depend on the judgment of the practitioner and are peculiar to each individual.

A. Enteral Administration

The active compounds of the present invention can advantageously be formulated for enteral administration, e.g., formulated for oral administration. The pharmaceutical forms may include sesame oil, peanut oil or aqueous propylene glycol; and sterile powders for the extemporaneous preparation of ingestible compositions, including tables, pills and capsules. Also, it is contemplated that the agents of the present invention can be provided in the form of a food additive and incorporated into a daily dietary program. All of these forms are generally selected to be sterile and stable under the conditions of manufacture and storage.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, paiabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the particular methods of preparation are vacuum-drying and freeze-drying techniques which yield a powder of the active ingredient plus any additional desired ingredient from a previously sterile-filtered solution thereof.

B. Other Routes of Administration

In addition to the compounds formulated for enteral administration, parenteral formulations such as intravenous or intramuscular injection are envisioned. Administration may also be nasal, buccal, rectal, vaginal or topical. Alternatively, administration may be by intradermal, subcutaneous, or intraperitoneal injection. The injection can be general, regional, local or direct injection, for example, of a tumor. Also contemplated is injection of a resected tumor bed, and continuous perfusion via catheter. Such compositions would normally be administered as pharmaceutically acceptable compositions, described supra.

### 6. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Cells injection. Three-month-old male Fischer 344 rats (250-350 g) were fed a choline-deficient diet. Each animal was anesthetized (3% isoflurane at 2.5 L/mn oxygen) and placed on a stereotaxic device (Stoelting, USA). The skin of the head was cleaned and incised. A hole was drilled through the skull 2 mm anterior and 2 mm lateral to the bregma, in the right-hand side of the skull. C6, 9L/LacZ or F98 cells (10⁶ in 10 μL cell culture media) in a ultra-low gelling temperature agarose are injected in the cortex at a 3 mm-depth at a rate of 2 μL/mn. A waiting time of 2 min was implemented following injection and bone wax was put in the burr-hole to prevent any reflux. The wound was sutured and covered with surgical glue. The surgery was performed in sterile conditions. The syringes containing the C6, 9L/LacZ or F98 cells are kept at 37° C. until the moment of use.

PBN treatment. Some rats were treated with PBN at a concentration of 0.065% (75 mg/kg/day) administered in the drinking water. The treatment was continuous and was started at different time points. The rats which received an injection of C6 cells were treated for the first time on the day of the surgery, 5 days before (n=3) or 5 days after. Some rats were not treated and served as controls (n=5). For the 9L/LacZ cells injections, 2 rats were not treated and 2 rats were administered PBN 5 days before the surgery.

MRI experiments. MRI experiments were performed on a 7 Tesla-30 cm horizontal bore magnet (Bruker BioSpin MRI Gmbh, Germany) at day 6-10 after the cells were injected and then every 2-3 days until the death of the rat. The animal was anesthetized (1.5% isoflurane at 1.5 L/mn oxygen) and placed in a MR probe (Bruker BioSpin MRI Gmbh, Germany). A head coil (Bruker BioSpin MRI Gmbh, Germany) was put on its head and the brain was localized.

Tumor growth monitoring. T1/T2-weighted images were obtained by a double-echo multi-slice spin echo method. Twenty-four axial slices of 1 mm thickness with an interslice distance of 1 mm were taken with a repetition time of 2369.64 ms and echo times of 17.41 ms and 63.85 ms for an acquisition time of 20 min. Tumor volumes were determined with the software ImageJ 1.32j (NIH, USA).

Angiography. Coronal slices are acquired by a FLASH (fast low angle shot) method with a volume of interest of 2.7×1.7×1.2 cm³, an echo time of 2.3 ms, a repetition time of 25 ms and a flip angle of 25° for an acquisition time of 27 min. A MIP (maximum intensity projection) technique was then applied to the data to obtain a 3D-angiography. Angiography was used to visualize new blood vessel formation, i.e., angiogenesis.

MRS experiments. Water suppression was performed by a CHESS (chemical shift suppression) method. A spectrum was acquired in a 4×4×4 cm³ voxel in the tumor region and in the contralateral control side of the brain, by using PRESS (point resolved spectroscopy) with a repetition time of 3000 ms, an echo time of 21.34 ms for an acquisition time of 13 min. An outer volume suppression tool was used to get rid of the 5 mm thick surrounding signal around the voxel. The metabolite peaks were assigned as follows, based on previous literature (Fan et al., 2004; Griffin et al., 2003): NAA at 2.02 ppm, Cr at 3.03 ppm, Cho at 3.25 ppm and mobile lipids at 1.33 ppm (methylene group) and 0.9 ppm (methyl group). Peak areas were calculated in reference to Cr peak and relative ratios were determined: NAA/Cho, NAA/Cr, Cho/Cr and Lipids/Cr. A decrease in NAA levels and an increase in lipids are indicative of the presence of a glioma (Ishimaru et al., 2001).

### Example 2

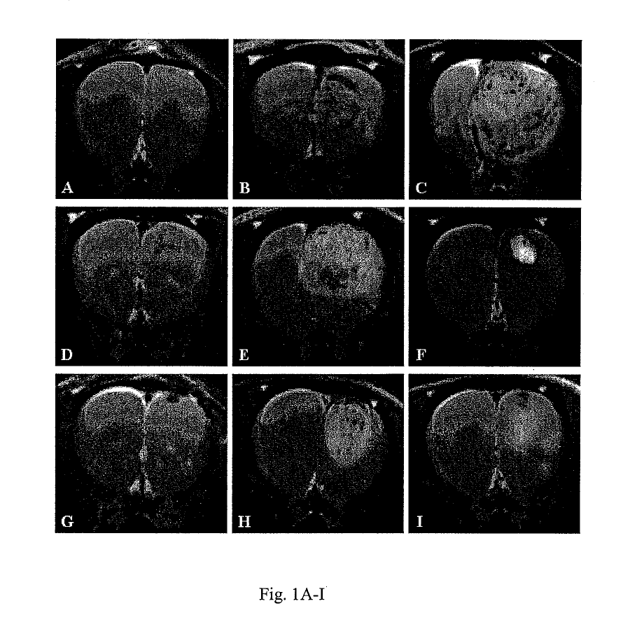

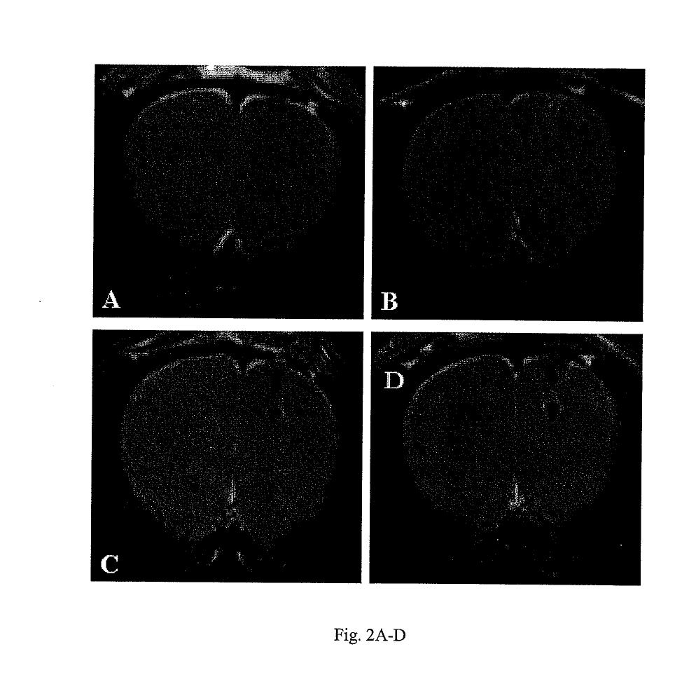

Tumor growth monitoring. In FIGS. 1A-I, the “normal” exponential growth of the C6 cells for the control rat is shown. PBN treatment started on the day of the surgery or 5 days after does not prevent, initially, tumor growth and shows the same doubling time (2.3-2.4 days) as the control rat. But after having reached a maximal volume (between 150 and 250 mm³), the tumor starts to regress until it almost completely disappears. A PBN treatment started 5 days before the surgery was found to be the most efficient in this setting, providing a very slow tumor growth (doubling time of 4.1 days) and a maximum volume of only 50 mm³. Only 2 out of the 4 rats injected with the 9L/LacZ cells demonstrated a slow tumor growth (FIGS. 2A-D). This cell line seems to present some growth problems and another kind of aggressive tumor cells may be required (e.g., F98) for experiments comparing with the C6 cell line.

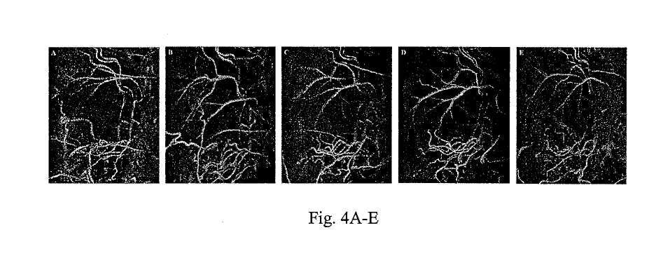

Angiography. The two cell lines show very different angiogenesis behaviors. C6 gliomas preferentially used the pre-existing blood vessels for their nutrient sources, i.e. the blood vessels appear wider than those in the brain of a normal rat (FIGS. 4C, D) and are sometimes displaced by the tumor mass (FIG. 4B). From day 6 after injection, 9L/LacZ cells quickly start to generate new blood vessels (FIG. 4E) even though the tumor size is only 5 mm ³. Noticeably, PBN treatment did not seem to induce changes in angiogenesis in the C6 gliomas. The next step is to superpose images of the tumors and corresponding angiograms, and estimate blood vessel volumes to monitor more precisely the angiogenesis process.

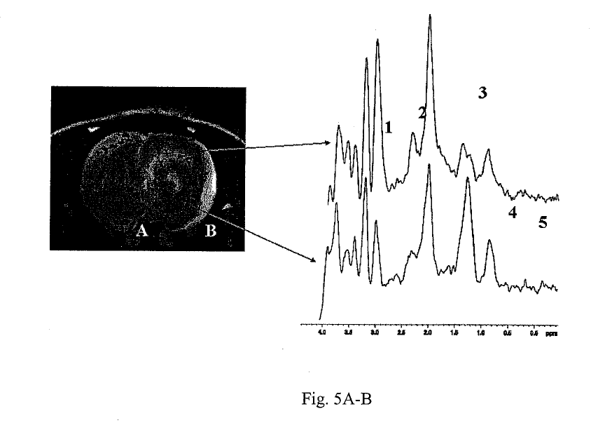

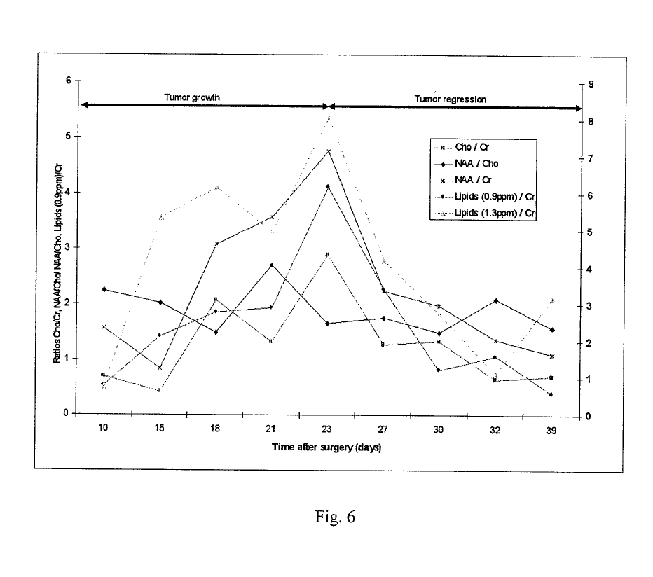

MRS experiments. During C6 glioma growth, Cho/Cr, NAA/Cr and especially Lipids/Cr ratios increased (FIGS. 5A, 5B, and 6) whereas the NAA/Cho ratio stayed relatively constant. When the tumor regresses, the ratios returned back to normal. It indicates that MRS is a useful method which can help in the monitoring of tumor growth, by following for example Lipids/Cr ratios. However, ratios are only surrogate markers of metabolite concentrations, i.e., for example both the levels of lipids and Cr change. The next step is to determine the absolute concentrations of each metabolite.

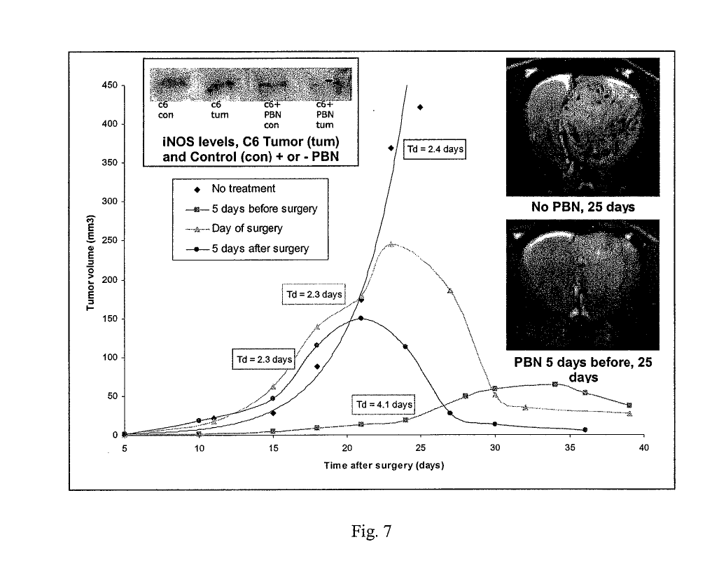

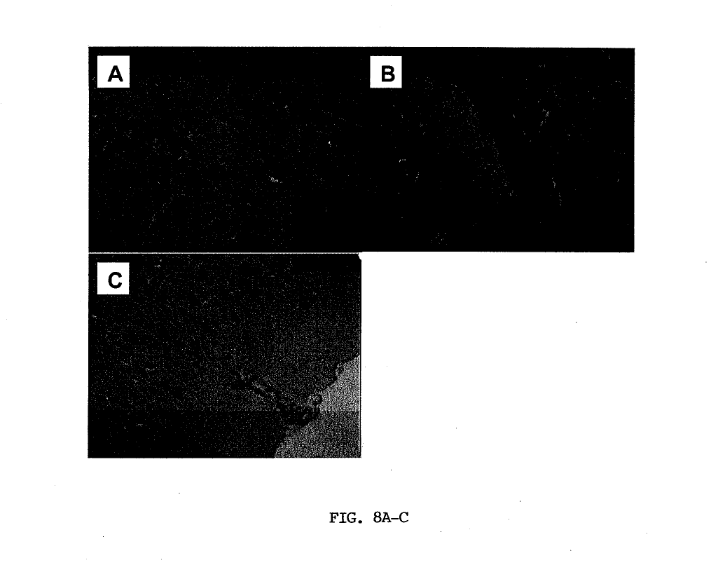

Conclusion. PBN treatment started 5 days before the injection of glioma cells was found to be the most efficient in inhibiting tumor growth (FIG. 7). FIGS. 8A-C show histology (H&E stain) slides of (FIG. 8A) control rat brain, (FIG. 8B) C6 rat glioma, (FIG. 8C) C6 rat glioma treated with PBN (5 days prior to implantation of cells), clearly indicating that PBN treated gliomas have no evidence of tumor cells. The malignant cells in the large neoplastic mass (FIG. 8B) were moderately atypical in appearance (grade II to III) with few mitotic and apoptotic figures being present. Large central areas of the mass 1c) were also necrotic.

PBN clearly modifies the morphology of the tumor and its growth rate. Its effects on the angiogenesis process and the metabolite changes still need to be further assessed. C6 and 9L/LacZ gliomas show very different angiogenesis behaviors as revealed by MR angiography, which could be a useful method to differentiate gliomas in human diagnosis. Changes in the metabolite ratios followed tumor growth and regression very closely, and can also be additionally used as a method to characterize gliomas in humans.

### Example 3

### Anti-Glioma Therapy Using PBN Treatment of Gliomas

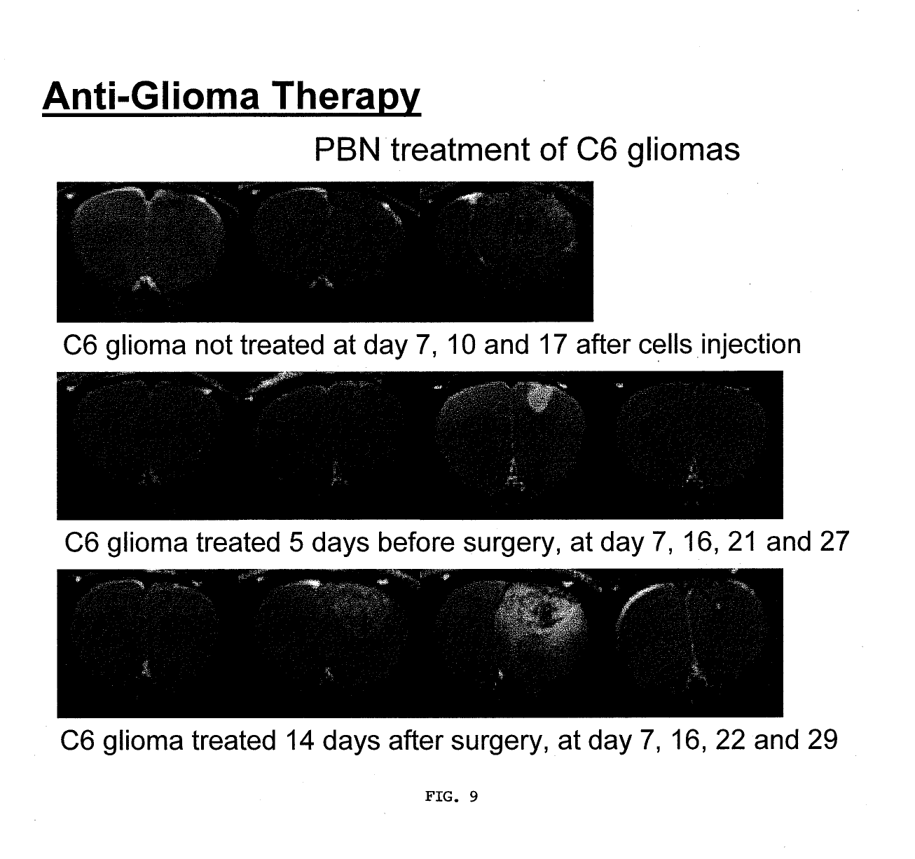

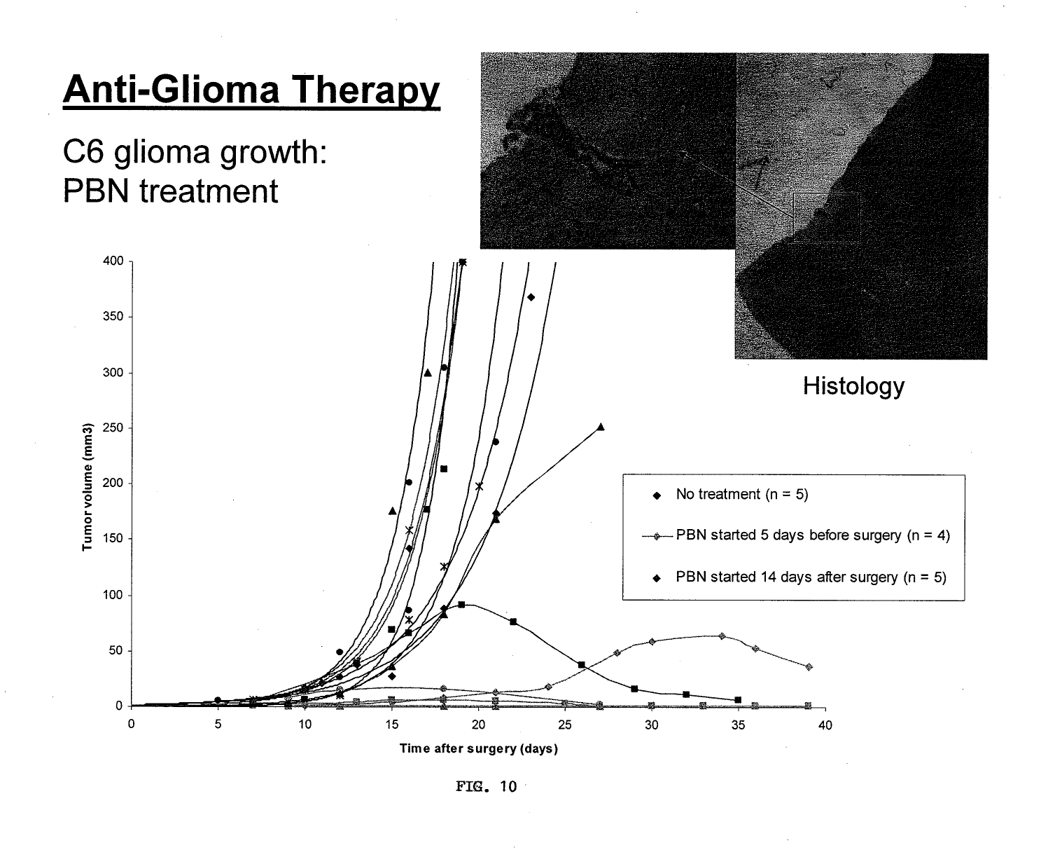

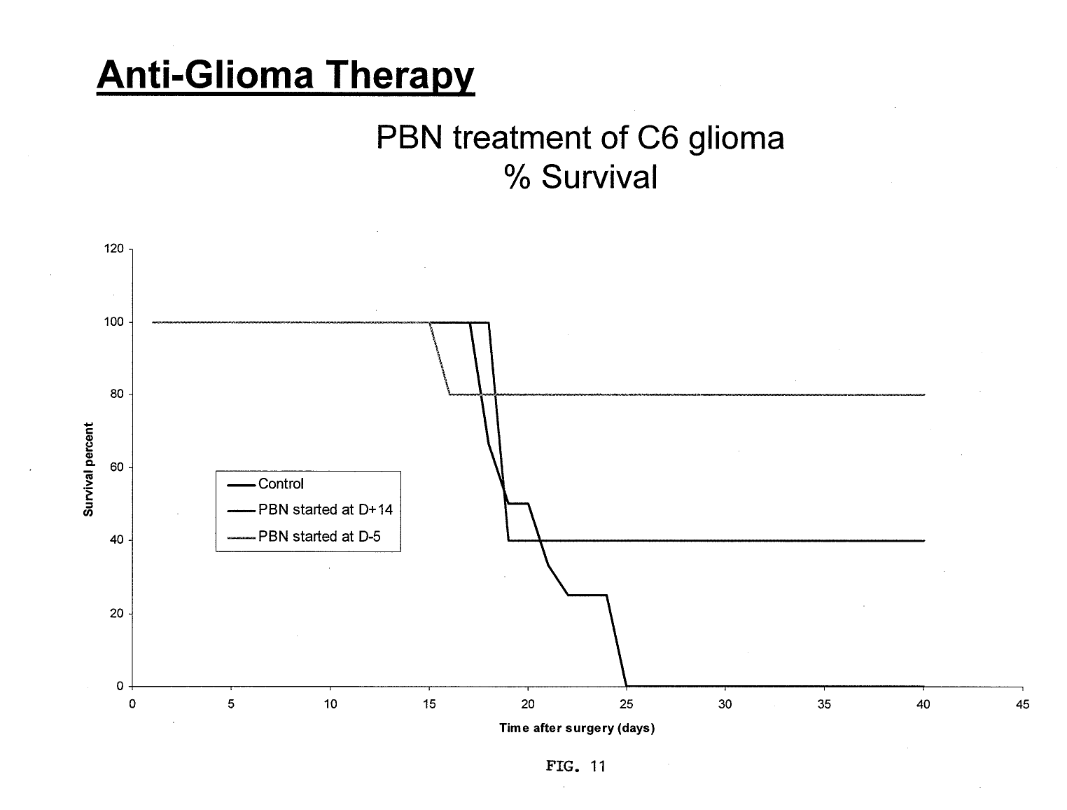

FIG. 9 shows T2-weighted MR images of C6 gliomas (top series) without PBN treatment at days 7, 10 and 17 after intracerebral cell implantation; (middle series) continuous PBN treatment starting 5 days prior to cell implantation at days 7, 16, 21 and 27; and (bottom series) continuous PBN treatment starting 14 days after cell implantation (when the tumor was >50 mm³) at days 7, 16, 22 and 29. A T2-weighted morphological MRI method was used. The results indicate that tumor growth was suppressed and that the tumor would recede if PBN was administered prior to or after tumor cell implantation. If PBN was given prior to cell implantation, glioma growth was suppressed, and recession to no tumor in all animals studied was observed (n=5). If PBN was administered after glioma formation has started (clinical relevant model), it results in recession of tumor growth (FIG. 9) and eradication of further tumor formation in 40% of animals treated (FIGS. 10 and 11, below).

FIG. 10 shows growth curves for therapy of C6 gliomas using PBN. Tumor growth (tumor volumes in mm³) curves for C6 gliomas of non-PBN treated (closed diamonds), PBN treatment 5 days prior to cell implantation (closed squares), and PBN treatment 14 days after cell implantation (closed triangles). Method used was NIH ImageJ calculation of tumor volumes from T2-weighted MR image slices of entire C6 tumors without or with PBN treatment. The results indicate that all non-PBN treated rats develop gliomas from days 10-12 and that tumors reach maximum sizes at days 17-24. C6 gliomas were found to either not develop or recede once intially formed in all rats treated with PBN 5 days prior to intracerebral cell implantation. C6 gliomas were found to recede in 40% of rats treated with PBN 14 days after cell implantation, i.e., once tumors were initially >50 mm3 in size. FIG. 11 shows % survival data for rats not treated with PBN, treated with PBN five days prior to intracerebral cell implantation, and 14 days after cell implantation based on survival data obtained from FIG. 10. The results indicate that non-PBN treated rats do not survive beyond 25 days after intracerebral C6 glioma implantion. Eighty percent of rats treated with PBN 5 days prior to C6 cell implantation survive longer than 40 days (study end-point), and 40% of rats treated with PBN fourteen 4 days after C6 cell implantation survive longer than 40 days.

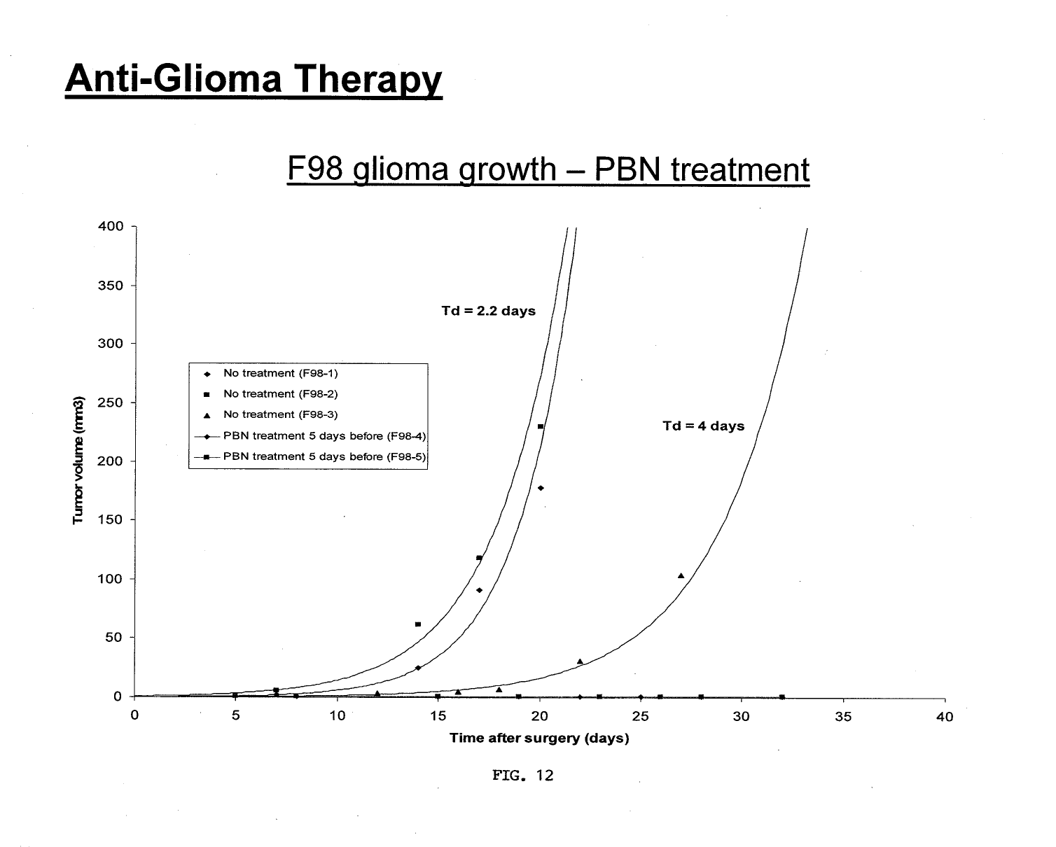

FIG. 12 shows tumor growth (tumor volumes in mm³) curves for F98 gliomas (low grade) of non-PBN treated, and PBN treatment 5 days prior to cell implantation. The method used was NIH ImageJ calculation of tumor volumes from in vivo T2-weighted MR image slices of entire F98 tumors without or with PBN treatment. The results indicate that all non-PBN treated rats develop gliomas from days 10-12 and that tumors reach maximum sizes at days 19-20. F98 gliomas were found to not develop in all rats treated with PBN five days prior to intracerebral cell implantation.

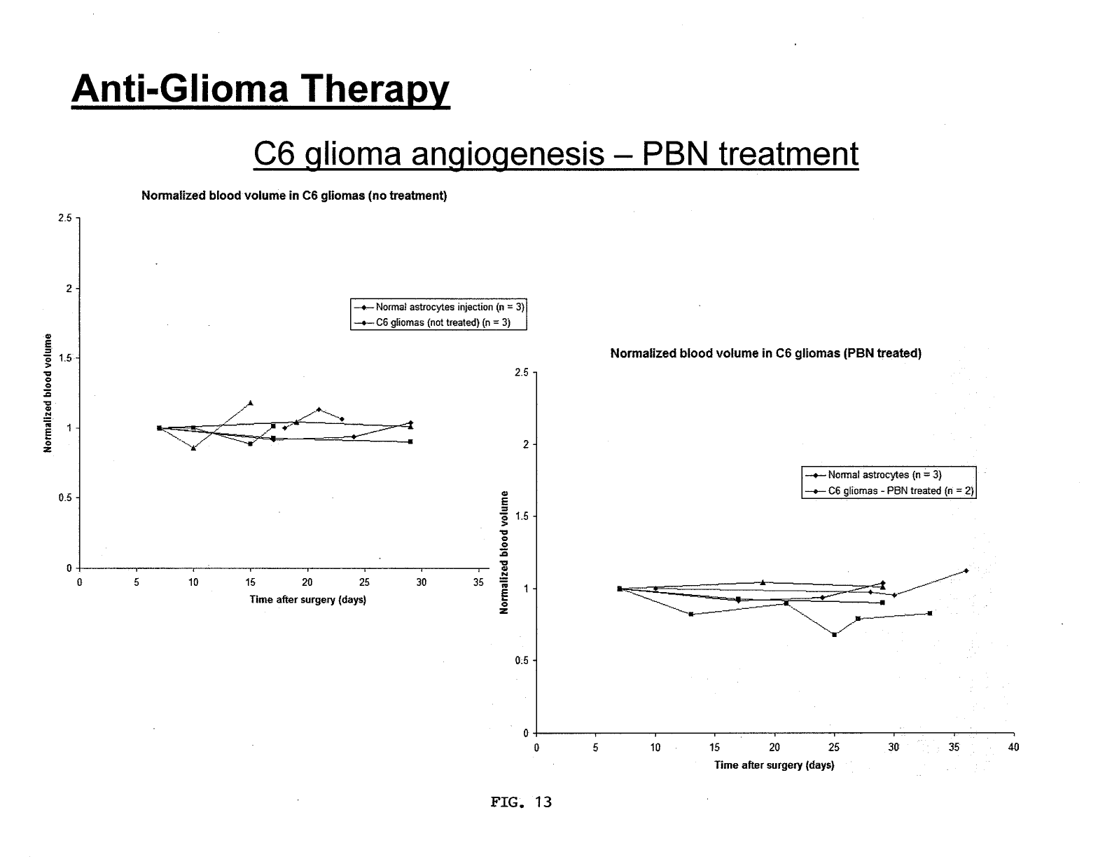

FIG. 13 shows the effect of PBN treatment of C6 glioma on angiogenesis. Normalized (compared to contralateral control side of brain) blood volumes from (left hand panel) non-PBN and (right hand panel) PBN treated (5 days prior to C6 cell implantation) rat brains in glioma regions. The method used was quantitative analysis (using a Mathematica-based program developed in our laboratory) of arterial signal intensities from in vivo MR angiography data. Glioma region arterial blood volumes were compared to blood volumes in the contralateral control cerebral hemisphere. The results indicate that tumor blood volumes increase in non-PBN treated C6 gliomas indicating angiogenesis, whereas PBN treatment 5 days prior to intracerebral C6 cell implantation results in suppression and/or a decrease in blood volumes.

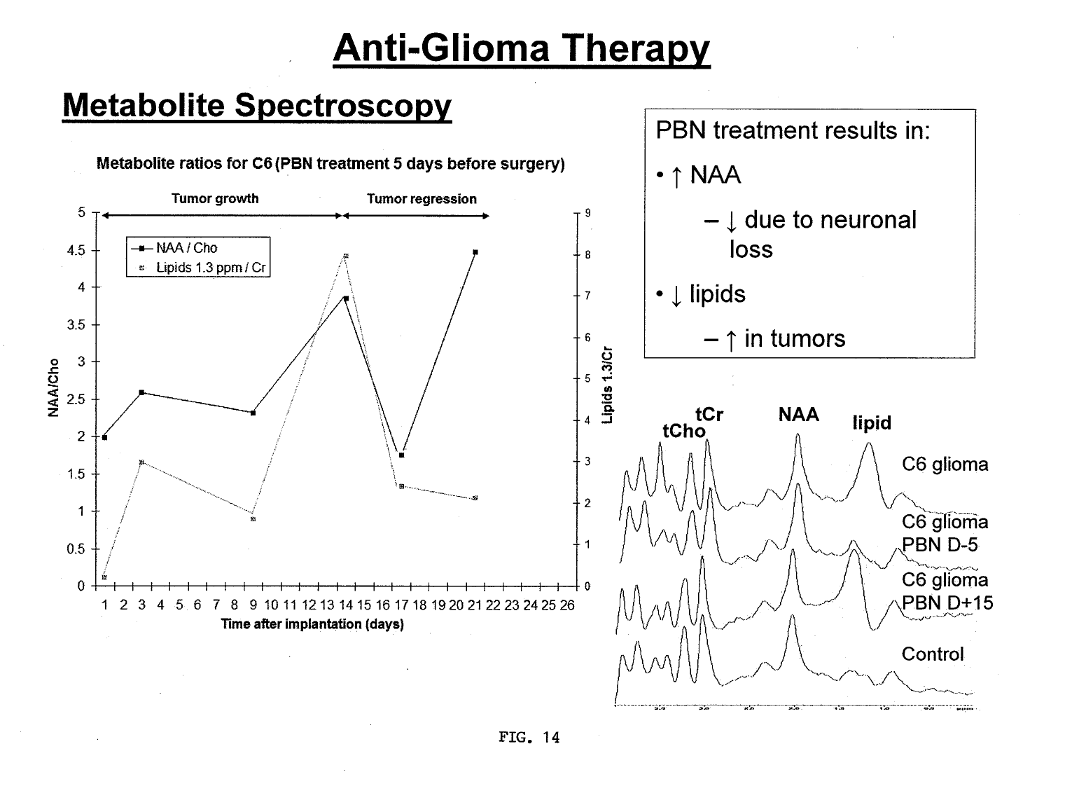

FIG. 14 shows metabolite spectroscopy. (Right hand panel) Image-guided MR spectroscopy in glioma tumor regions in non-PBN (C6 glioma) and PBN treated (C6 glioma PBN D-5 (PBN administed 5 days prior to cell implantation), and C6 glioma PBN D+15 (PBN administered 14-15 days after cell implantation) rats, compared to a normal brain region (Control). (Left hand panel) N-acetyl aspartate (NAA) to choline (Cho), and Lipids (at 1.3 ppm) to creatine (Cr) metabolite level ratios of a C6 glioma treated 5 days prior to cell implantation. Method used was image-guided MR spectroscopy in a volume of 125 mm³ or microliters in selected regions of control or tumor regions in rats in vivo. MR spectral peak areas were measured using the Bruker XWIN NMR peak intragration software package. The results indicate that NAA (a marker of neuronal cell viability) levels (compared to choline (Cho) levels) increase to normal levels, and that lipid (a marker of tumor growth) levels (compared to creatine (Cr) levels) decrease to normal levels, when rats are treated with PBN.

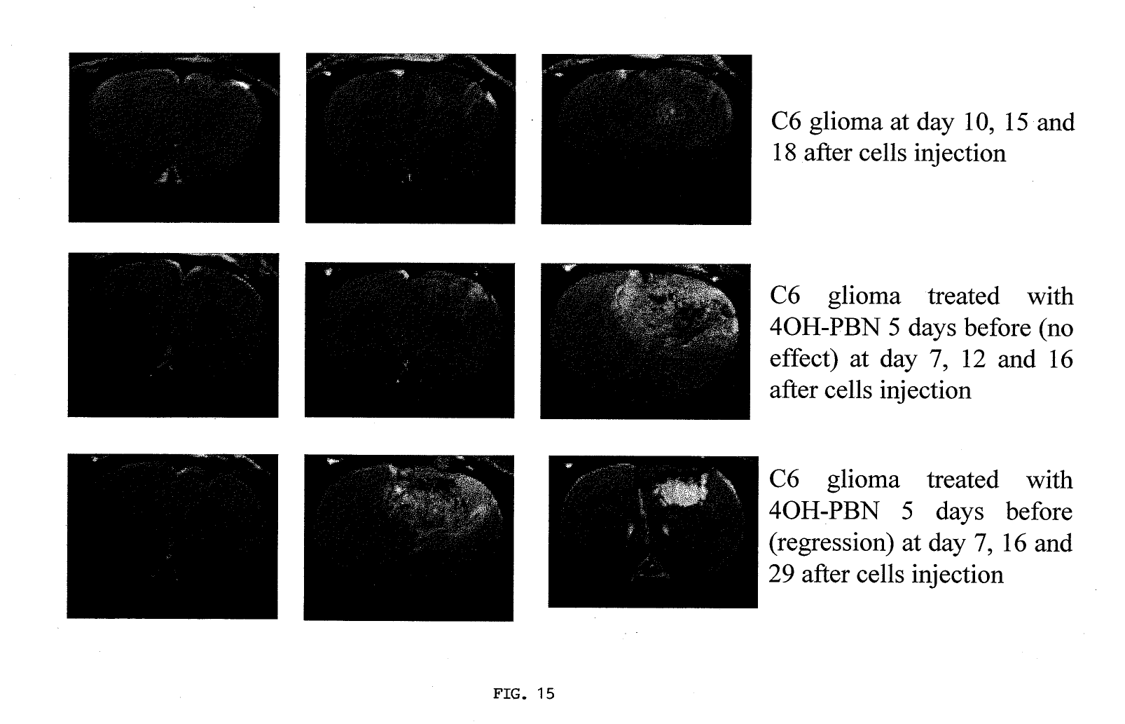

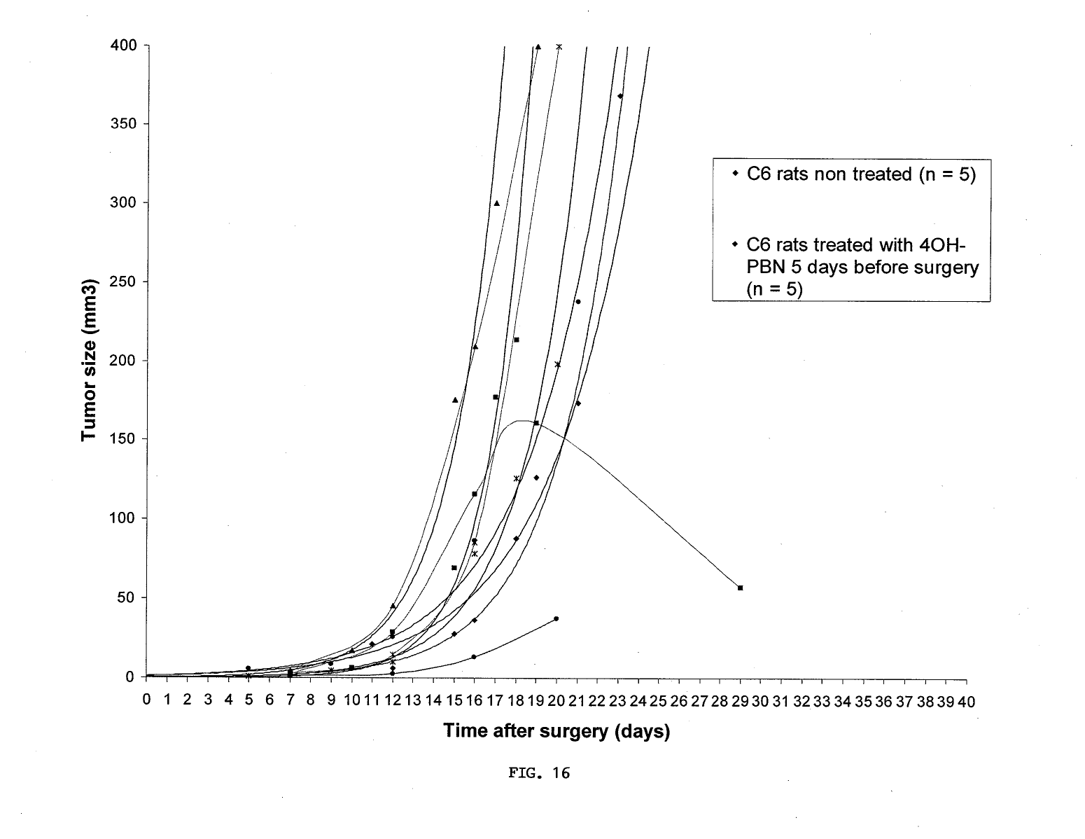

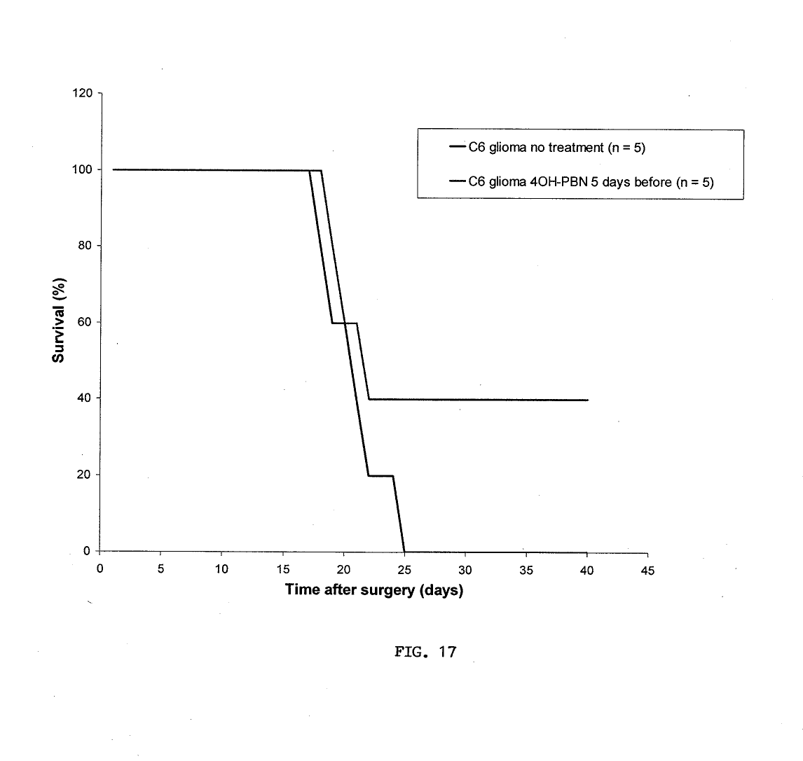

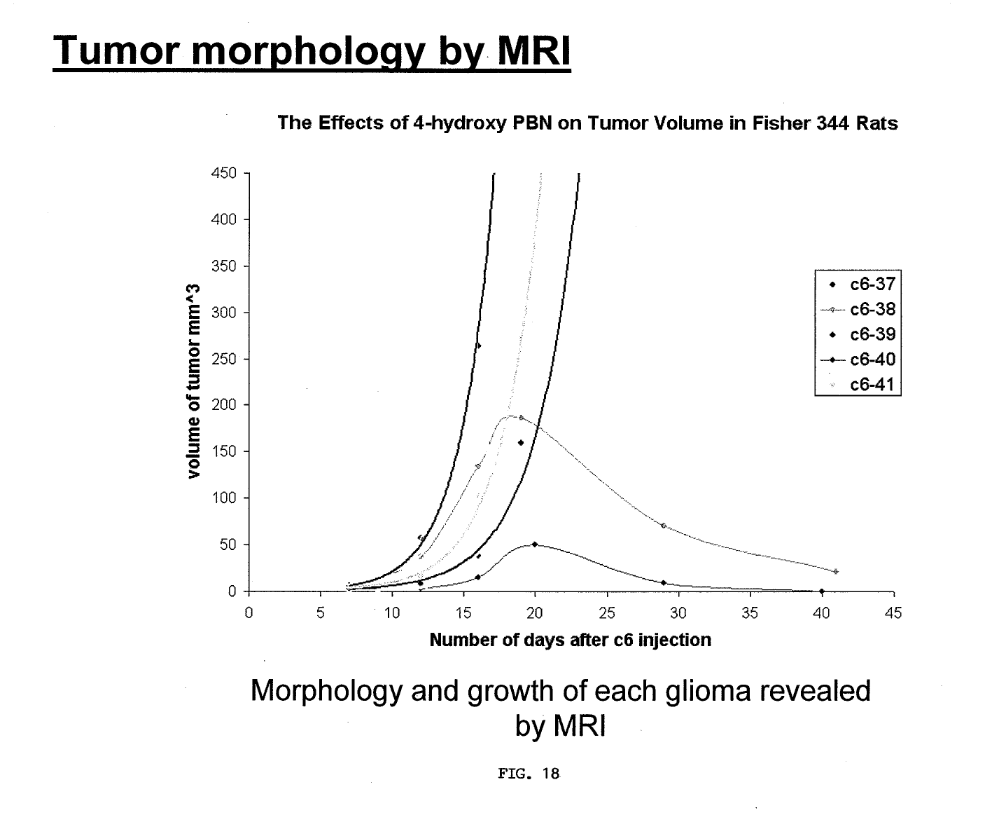

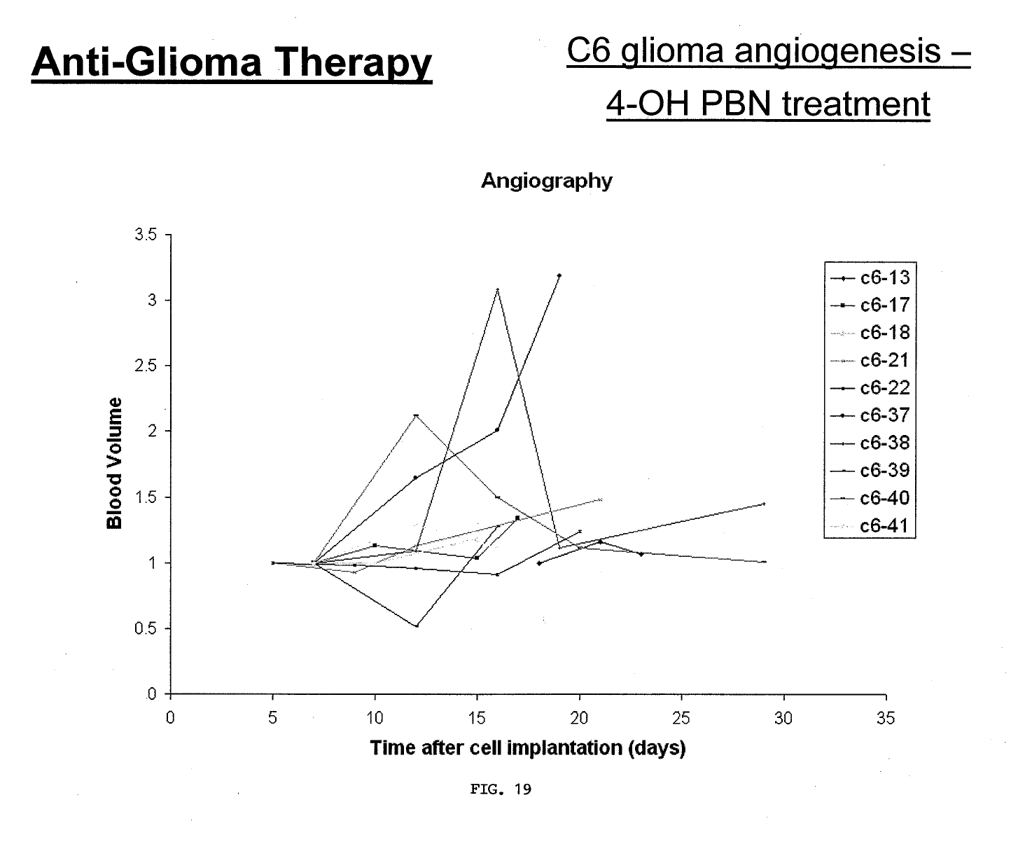

FIG. 15 shows T2-weighted MRI of C6 glioma cell intracerebral implantations with 4-hydroxy-PBN, the main metabolite of PBN, administered continuously in drinking water (75 mg/kg/day) 5 days prior to C6 cell implantation. 4-hydroxy-PBN was found to suppress and recess tumor growth in 40% of animals tested (n=5). FIG. 16 shows tumor volumes measured by NIH Image J software. 4-hydroxy-PBN was found to suppress and recess tumor growth in 40% of animals tested (n=5). FIG. 17 shows that 4-hydroxy-PBN suppressed and recessed tumor growth in 40% of animals tested (n=5). FIGS. 18 and 19 show morphology data and angiography data, respectively, for 4-hydroxy-PBN treated C6 gliomas. Decrease in tumor growth and angiogenesis was found in 2 of 5 animals treated.

### Example 4

### PBN and HIF-1

As discussed above, nitrones have been shown to be potent anti-cancer agents. In particular, the inventors have shown that PBN is potently effective in preventing the development of gliomas and glioblastomas in rat models. Mechanistic studies have implied that PBN's activity is strongly coupled to its ability to suppress the induction of inducible nitric oxide synthase (iNOS) therefore preventing higher levels of nitric oxide (NO) formation in the target tissue. The inventors' interpretation of the many studies in several human tumors as well as animal models of cancers clearly demonstrate that NO produced by iNOS at modest levels is a very important agent that enhances the continued growth and development of tumors. PBN acts to inhibit tumor growth and development by preventing NO formation, specifically by inhibiting the expression of iNOS. The inventors' background studies provide a strong scientific mechanistic basis to explain why iNOS/NO is important in tumor development and explains why specific nitrones are expected to be effective anti-cancer agents with significant commercial potential.

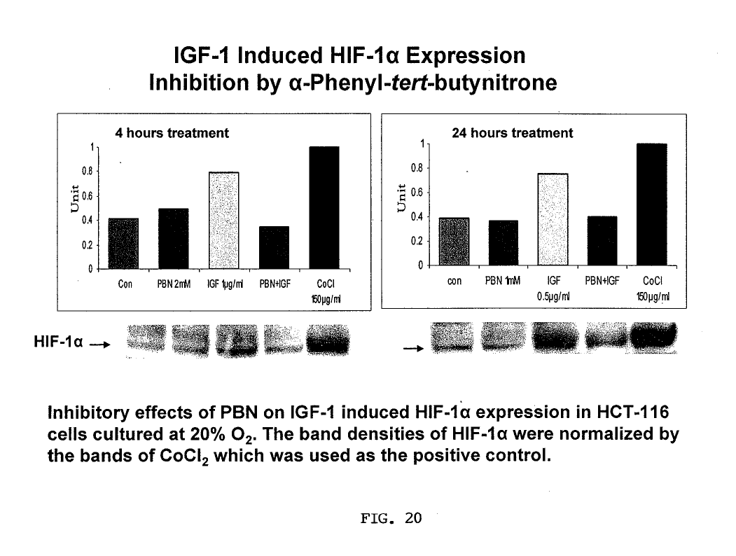

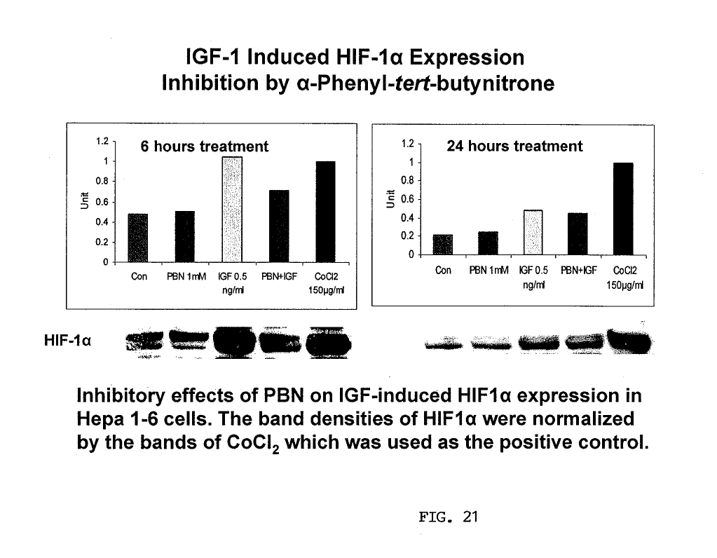

Recently, the inventors have shown that PBN inhibits c-MET, a tyrosine kinase inhibitor, associated with invasive cell growth. In addition, very recent results have shown that PBN inhibits the induction of HIF-1α in several cancer cells. HIF-1α is a major transactivation factor which is central to the induction of VEGF as well as iNOS and many other genes very important in the development of cancer. VEGF is very important in angiogenesis essential for tumor growth and is a major target of several anti-cancer companies. Therefore, observations of PBN inhibition of HIF-1α induction and/or c-MET (associated with HIF-1α) implicate that these compounds may be active in a wide range of cancers and that our approach is novel. Experimental results demonstrating that PBN inhibits the induction of HIF-1α in an aggressive human colon cancer cell line, HCT116, is presented in FIG. 20. FIG. 21 shows that PBN inhibits the induction of HIF-1α in a mouse hepatocellular carcinoma cell line.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,859,452
U.S. Pat. No. 4,925,835
U.S. Pat. No. 5,569,902
Bardos and Ashcroft, Biochim. Biophys. Acta, 1755:107-120, 2005.
Bigner et al, In: Pathology of Tumors of the Nervous System, Russell and Rubinstein (Eds.), 6th Edition, London:Edward Arnold, 757, 1998.
Burger et al., In: Surgical Pathology of the Nervous System and Its Coverings, 3^{rd} ed., New York, Churchill Livingstone, Inc, 737, 1991.
Burger et al, Cancer, 56:1106-1111, 1985.
Burger etal., Cancer, 59:1345-1352, 1987.
Cao et al., Brain Res., 644:267-272, 1994.
Carney et al., Proc. Natl. Acad. Sci. USA, 88:3633-3636, 1991.
Chau et al., Cancer Res., 65:4918-4928, 2005.
Clough-Helfman et al., Free Radic. Res. Commun., 15:177-186, 1991.
Csiki etal., Cancer Res., 66:143-150,2006.
Dann and Bruick, Biochem. Biophys. Res. Commun., 338:639-647, 2005.
Daumas-Duport et al., J. Neurooncol., 34:61-78, 1997.
Davis et al., J. Neurooncol., 24:9-12, 1995.
Dehghani et al., Acta Neuropathol., 95:493-504, 1998.
Dimova et al., Thromb. Haemost., 93:1176-1184, 2005.
Ernestus et al., Acta. Histochem. Suppl., 42:159-164, 1992.
Ernestus et al., J. Neurooncol., 29(2):167-174, 1996.
Fan et al., Clinical Radiology, 59:77, 2004.
Floyd et al., Biochem. Biophys. Res. Commun., 74:79-84, 1977.
Floyd et al., FASEB J., 4:2587-2597, 1990.
Floyd et al., In: Neuroprotective Approaches to the Treatment of Parkinson's Disease and other Neurodegenerative Disorders, Chapman et al. (Eds.), Academic Press Limited, London, 69-90, 1996.
Floyd, Adv. Pharmacol., 38:361-378, 1997.
Floyd, Aging Cell, 5:51-57, 2006.
Folbergrova et al., Proc. Natl. Acad. Sci. USA, 92:5057-5061, 1995.
Fukuda et al., J. Biol Chem., 277:38205-38211, 2002.
Gradin et al., J. Biol Chem., 277:23508-23514, 2002.
Gradin et al., Mol. Cell Biol., 16:5221-5231, 1996.
Griffin et al., Cancer Research, 63:3195, 2003.
Heegard et al., Cancer, 76:1809-1813, 1995.
Hensley etal., Anal. Biochem., 251:187-195, 1997.
Hensley et al., In: Neuroprotective Agents and Cerebral Ischaemia, Green and Cross (Eds.), Academic press Ltd., London, 299-317, 1996.
Hensley et al., J. Neurochem, 71:2549-2557, 1998.
Hofer et al., FASEB J., 15:2715-2717, 2001.
Hoshino et al., Int. J. Cancer, 53:550-555, 1993.
Huang etal., J. Biol. Chem., 271:32253-32259, 1996.
Ibrahim et al., Cancer Res., 65:11094-11100, 2005.
Isaacs et al., J. Biol. Chem., 277:29936-29944, 2002.
Ishimaru et al., Eur. Radiol., 11: 1784-1791, 2001.
Janzen, Acc. Chem. Res., 4:31-40, 1971.
Kaelin, Jr., Biochem. Biophys. Res. Commun., 338:627-638, 2005.
Kim et al., Int J. Oncol., 26:1049-1052, 2005.
Kleihues and Cavenee, In: Pathology and Genetics of Tumors of the Nervous System, IARC Press, Lyon, 227-228, 2000.
Kleihues and Ohgaki, Brain Pathol, 7:1131-1136, 1997.
Kleihues and Ohgaki, Neuro-Oncology, 1:44-51, 1999.
Kleihues et al., In: Histological Typing of Tumours of the Central Nervous System, 2^{nd} Ed., Berlin: Springer-Verlag, 112, 1993.
Kotake, Antioxid. Redox. Signal, 1:481-499, 1999.
Kros etal., Cancer, 78:1107-1113, 1996.
Kubo et al., Biochem. Biophys. Res. Commun., 318:1006-1011, 2004.
Kwon et al., Clin. Cancer Res., 11:7607-7613, 2005.
Lamborn et al., Cancer, 85:925-935, 1999.
Levin and Prados, J. Clin. Oncol., 10(5):766-71, 1992.
Levin et al., Clin. Cancer Res., 6(10):3878-3884, 2000.
Levin et al., Int. J. Radiat. Oncol. Biol Phys., 32(1):75-83, 1995.
Mabjeesh et al., Cancer Res., 62:2478-2482, 2002.
Martinez-Ruiz et al., Proc. Natl. Acad. Sci. USA, 102:8525-8530, 2005.
Maxwell, Semin. Cell Dev. Biol., 16:523-530, 2005.
Minet et al., FEBS Lett., 460:251-256, 1999.
Minet et al, Free Radic. Biol. Med., 31:847-855, 2001.
Miyajima et al., Biochem. Biophys. Res. Commun., 215:114-121, 1995.
Muller et al., Acta Neurochir (Wien), 37:75-91, 1977.
Nelson et al., Cancer, 52:550-554, 1983.
Nikinmaa et al., J. Cell Sci., 117:3201-3206, 2004.
Novelli et al., In: Oxygen Free Radicals in Shock, Novelli and Ursini (Eds.), Karger, Basel, 119-124, 1986.
Pahlmark et al., Acta Physiol. Scand., 157:41-51, 1996.
Pogrebniak et al., Surgery, 112:130-139, 1992.
Poyer et al., Biochim. Biophys. Acta, 539:402-409, 1978.
Prados et al., Neurosurgery, 24(6):806-809, 1989.
Richard et al., J. Biol Chem., 274:32631-32637, 1999.
Sang et al., Arch. Biochem. Biophys. 363:341-348, 1999.
Scalabrino and Ferioli, Cancer Detect. Prev., 8(1-2):11-16, 1985.
Scalabrino et al., J. Natl. Cancer Inst., 68(5):751-754, 1982.
Semenza and Wang, Mol. Cell Biol, 12:5447-5454, 1992.
Semenza, et al., J. Biol. Chem., 269:23757-23763, 1994.
Shaw et al., Neurosurgery, 34:577-582, 1994.
Tabatabaie et al., Biochem. Biophys. Res. Commun., 221:386-39, 1996.
Vaupel,. Oncologist, 9(5):10-17, 2004.
Wacker et al., J. Neuro-Oncology, 19:113-122, 1994.
Wang and Semenza, Proc. Natl. Acad. Sci. USA, 90:4304-4308, 1993a.
Wang and Semenza, J. Biol Chem., 268:21513-21518, 1993b.
Wang and Semenza, J. Biol. Chem., 270:1230-1237, 1995.
Wang et al., Proc. Natl. Acad. Sci. USA, 92:5510-5514, 1995.
Welsh et al., Cancer Res., 62:5089-5095, 2002.
Welsh et al., Mol Cancer Ther., 2:235-243, 2003.
Welsh et al., Mol Cancer Ther., 3:233-244, 2004.
Zundel et al, Genes Dev., 14:391-396, 2000.

What is claimed is:

1. A method for inhibiting the vascularization, growth or spread of a glioma comprising administering to a human subject with glioma a dose of a nitrone free radical trapping agent effective to inhibit the vascularization, growth or spread of said glioma.

2. The method of claim 1, wherein the nitrone free radical trapping agent is an N-alkyl nitrone free radical trapping agent.

3. The method of claim 1, wherein the agent is phenyl N-tert-butylnitrone, 3-hydroxyphenyl N-tert-butylnitrone, 2-hydroxyphenyl N-tert-butylnitrone, 2-sulfoxyphenyl N-tert-butylnitrone or 4-hydroxyphenyl N-tert-butylnitrone, or derivatives thereof.

4. The method of claim 1, wherein the human subject has a recurrent or metastatic glioma.

5. The method of claim 1, wherein the human subject has previously failed one or more anti-glioma therapies.

6. The method of claim 1, wherein the effective dose is from about 5 to about 150 mg/kg body weight per day.

7. The method of claim 1, wherein administering is through dietary administration, oral administration or via intravenous injection.

8. The method of claim 7, wherein the oral administration is in the form of a pill or a liquid.

9. The method of claim 7, wherein the intravenous injection is in the form of a mixture containing an injectable vehicle.

10. The method of claim 7, wherein the dietary administration is through supplementation of a food component.

11. The method of claim 10, wherein the effective amount is from about 0.005 w/w % to about 0.1 w/w % of the diet being administered.

12. The method of claim 1, wherein said glioma is an astrocytoma, an oligodendroglioma, or a glioblastoma multiforme.

13. A method for inhibiting glioma development comprising (a) identifying a human subject at risk of developing a glioma and (b) administering to said subject a dose of a nitrone free radical trapping agent effective to inhibit the development of said glioma.

14. The method of claim 13, wherein the nitrone free radical trapping agent is an N-alkyl nitrone free radical trapping agent.

15. The method of claim 13, wherein the agent is phenyl N-tert-butylnitrone, 3-hydroxyphenyl N-tert-butylnitrone, 2-hydroxyphenyl N-tert-butylnitrone, 2-sulfoxyphenyl N-tert-butylnitrone or 4-hydroxyphenyl N-tert-butylnitrone, or derivatives thereof.

16. The method of claim 13, wherein the human subject has a familial history of cancer or has been exposed to a carcinogenic environment.

17. The method of claim 16, wherein specific glioma risk factors include exposure to N-nitroso compounds or X-irradiation.

18. The method of claim 13, wherein the effective dose is from about 5 to about 150 mg/kg body weight per day.

19. The method of claim 13, wherein administering is through dietary administration.

20. The method of claim 19, wherein the dietary administration is through supplementation of a food component.

21. The method of claim 20, wherein the effective amount is from about 0.005 w/w % to about 0.1 w/w % of the diet being administered.

22. The method of claim 13, wherein said glioma is an astrocytoma, an oligodendroglioma, or a glioblastoma multiforme.

23. A method for inhibiting glioma recurrence comprising administering to a subject previously having a glioma a dose of a nitrone free radical trapping agent effective to inhibit the development of said glioma.

24. The method of claim 23, wherein the nitrone free radical trapping agent is an N-alkyl nitrone free radical trapping agent.

25. The method of claim 23, wherein the agent is phenyl N-tert-butylnitrone, 3-hydroxyphenyl N-tert-butylnitrone, 2-hydroxyphenyl N-tert-butylnitrone, 2-sulfoxyphenyl N-tert-butylnitrone or 4-hydroxyphenyl N-tert-butylnitrone, or derivatives thereof.

26. The method of claim 23, wherein said glioma is an astrocytoma, an oligodendroglioma, or a glioblastoma multiforme.

27. The method of claim 23, wherein the effective dose is from about 5 to about 150 mg/kg body weight per day.

28. The method of claim 1, further comprising measuring inducible nitric oxide synthase (iNOS) levels in cells of said glioma.

29. The method of claim 28, wherein measuring comprises MRI using a labeled anti-iNOS antibody.

30. The method of claim 1, further comprising measuring nitric oxide (NO) levels in tissues of said glioma.

31. The method of claim 30, wherein measuring comprises MRI using a NO spin trapping agent.

32. The method of claim 31, wherein said NO spin trapping agent is N-methyl-D-glucamine dithiocarbamate (MGD)-Fe(II)-NO complex.

33. The method of claim 1, further comprising a secondary anti-glioma therapy.

34. The method of claim 33, wherein the secondary anti-glioma therapy is chemotherapy.

35. The method of claim 34, wherein the chemotherapy is lomustine, vincristine, matulane, PCV, BCNU, CCNU and/or DFMO.

36. The method of claim 33, wherein the secondary anti-glioma therapy is radiation.

37. The method of claim 33, wherein the secondary anti-glioma therapy is surgery.
